SEQUENCE LISTING (32 sequences)

SEQ ID NO: 1 - protein, 249 aa, Artificial Sequence
  Artificially synthesized GA733 amino acid sequence
TATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 2 - DNA, 747 bp, Artificial Sequence
  Artificially synthesized GA733 nucleotide sequence
acggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaa

SEQ ID NO: 3 - protein, 30 aa, Artificial Sequence
  Artificially synthesized ER signal peptide
MATQRRANPSSLHLITVFSLLAAVVSAEVD

SEQ ID NO: 4 - protein, 192 aa, Artificial Sequence
  Artificially synthesized Fc fragment of IgG1 amino acid sequence
RTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 576 bp, Artificial Sequence
  Artificially synthesized Fc fragment of IgG1 nucleotide sequence
cggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctcgaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccagagaacaactacaagaccacgcctcccgtgctggactccgacggctccttcttcctctatagcaagctcaccgtggacaagagcaggtggcagcaggggaacgtcttctcatgctctgtgatgcatgaggctctgcacaaccactacacgcagaagagcctctccctgtccccgggt

SEQ ID NO: 6 - protein, 237 aa, Artificial Sequence
  Artificially synthesized Fc fragment of IgG1 containing hinge region amino acid sequence
AAADLVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - DNA, 711 bp, Artificial Sequence
  Artificially synthesized Fc fragment of IgG1 containing hinge region nucleotide sequence
gcggccgcagatctcgttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctcgaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccagagaacaactacaagaccacgcctcccgtgctggactccgacggctccttcttcctctatagcaagctcaccgtggacaagagcaggtggcagcaggggaacgtcttctcatgctctgtgatgcatgaggctctgcacaaccactacacgcagaagagcctctccctgtccccgggt

SEQ ID NO: 8 - protein, 4 aa, Artificial Sequence
  Artificially synthesized KDEL for ER retention signal amino acid sequence
KDEL

SEQ ID NO: 9 - protein, 520 aa, Artificial Sequence
  Artificially synthesized GA733-FcK amino acid sequence
MATQRRANPSSLHLITVFSLLAAVVSAEVDTATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAAADLVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDEL

SEQ ID NO: 10 - DNA, 1560 bp, Artificial Sequence
  Artificially synthesized GA733-FcK nucleotide sequence
atggctactcaacgaagggcaaaccctagctctctccatctaattactgtattctctctgctagctgctgtcgtctccgctgaggtcgacacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagcggccgcagatctcgttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctcgaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccagagaacaactacaagaccacgcctcccgtgctggactccgacggctccttcttcctctatagcaagctcaccgtggacaagagcaggtggcagcaggggaacgtcttctcatgctctgtgatgcatgaggctctgcacaaccactacacgcagaagagcctctccctgtccccgggtaaagatgaactc

SEQ ID NO: 11 - protein, 467 aa, Artificial Sequence
  Artificially synthesized heavy chain of CO17-1A amino acid sequence
MEWSRVFIFLLSVTAGVHSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDGPWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVKWTNNGKTELNYKNTEPVLDSDGSYFMYSKLRVEKKNWVERNSYSCSVVHEGLHNHHTTKSFSRTPGKID

SEQ ID NO: 12 - protein, 471 aa, Artificial Sequence
  Artificially synthesized heavy chain of CO17-1AK amino acid sequence
MEWSRVFIFLLSVTAGVHSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDGPWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVKWTNNGKTELNYKNTEPVLDSDGSYFMYSKLRVEKKNWVERNSYSCSVVHEGLHNHHTTKSFSRTPGKIDKDEL

SEQ ID NO: 13 - protein, 238 aa, Artificial Sequence
  Artificially synthesized light chain of CO17-1A or light chain of CO17-1AK amino acid sequence
MGIKMESQTLVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - DNA, 1401 bp, Artificial Sequence
  Artificially synthesized heavy chain of CO17-1A nucleotide sequence
atggaatggagcagagtctttatctttctcctatcagtaactgcaggtgttcactcccaggtccagttgcagcagtctggagctgagctggtaaggcctgggacttcagtgaaggtgtcctgcaaggcttctggatacgccttcactaattacttgatagagtgggtaaagcagaggcctggacagggccttgagtggattggggtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagagatggtccctggtttgcttactggggccaagggactctggtcactgtctctgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtgaagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaatcgat

SEQ ID NO: 15 - DNA, 1413 bp, Artificial Sequence
  Artificially synthesized heavy chain of CO17-1AK nucleotide sequence
atggaatggagcagagtctttatctttctcctatcagtaactgcaggtgttcactcccaggtccagttgcagcagtctggagctgagctggtaaggcctgggacttcagtgaaggtgtcctgcaaggcttctggatacgccttcactaattacttgatagagtgggtaaagcagaggcctggacagggccttgagtggattggggtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagagatggtccctggtttgcttactggggccaagggactctggtcactgtctctgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtgaagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaaatcgataaggacgaactt

SEQ ID NO: 16 - DNA, 717 bp, Artificial Sequence
  Artificially synthesized light chain of CO17-1A or light chain of CO17-1AK nucleotide sequence
atgggcatcaagatggaatcacagactctggtcttcatatccatactgctctggttatatggagctgatgggaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 17 - DNA, 27 bp, Artificial Sequence
  Artificially synthesized forward primer for GA733-FcK
gtcgacacggcgacttttgccgcagct

SEQ ID NO: 18 - DNA, 24 bp, Artificial Sequence
  Artificially synthesized reverse primer for GA733-FcK
gagttcatctttacccggggacag

SEQ ID NO: 19 - DNA, 21 bp, Artificial Sequence
  Artificially synthesized forward primer for heavy chain of CO17-1AK
atggaatggagcagagtcttt

SEQ ID NO: 20 - DNA, 21 bp, Artificial Sequence
  Artificially synthesized reverse primer for heavy chain of CO17-1AK
atcgattttacccggagtccg

SEQ ID NO: 21 - DNA, 21 bp, Artificial Sequence
  Artificially synthesized forward primer for light chain of CO17-1AK
atgggcatcaagatcgaatca

SEQ ID NO: 22 - DNA, 21 bp, Artificial Sequence
  Artificially synthesized reverse primer for light chain of CO17-1AK
acactcattcctgttgaagct

SEQ ID NO: 23 - protein, 4 aa, Artificial Sequence
  Artificially synthesized HDEL for ER retention signal amino acid sequence
HDEL

SEQ ID NO: 24 - protein, 6 aa, Artificial Sequence
  Artificially synthesized SEKDEL for ER retention signal amino acid sequence
SEKDEL

SEQ ID NO: 25 - protein, 5 aa, Artificial Sequence
  Artificially synthesized KHDEL for ER retention signal amino acid sequence
KHDEL

SEQ ID NO: 26 - protein, 4 aa, Artificial Sequence
  Artificially synthesized KEEL for ER retention signal amino acid sequence
KEEL

SEQ ID NO: 27 - protein, 6 aa, Artificial Sequence
  Artificially synthesized SEHDEL for ER retention signal amino acid sequence
SEHDEL

SEQ ID NO: 28 - protein, 32 aa, Artificial Sequence
  Artificially synthesized Arabidopsis derived ABPI endoplasmic reticulum targeting signal peptide
MIVLSVGSASSSPIVVVFSVALLLFYFSETSL

SEQ ID NO: 29 - protein, 24 aa, Artificial Sequence
  Artificially synthesized ER signal sequence of alpha carbonic anhydrase I gene (NP_850685)
MKIMMMIKLCFFSMSLICIAPADA

SEQ ID NO: 30 - protein, 24 aa, Artificial Sequence
  Artificially synthesized ER signal sequence from an unnamed protein (BAG87020)
MAASHGNAIFVLLLCTLFLPSLAC

SEQ ID NO: 31 - protein, 24 aa, Artificial Sequence
  Artificially synthesized ER signal sequence of ribophorin I (AT2G01720)
MAARIGIFSVFVAVLLSISAFSSA

SEQ ID NO: 32 - protein, 21 aa, Artificial Sequence
  Artificially synthesized ER signal sequence from A. thaliana basic chitinase
MKTNLFLFLIFSLLLSLSSAE

(19) United States
(12) Patent Application Publication
(10) Pub. No.: US 2017/0159066 A1
(43) Pub. Date: Jun. 8, 2017

(54) METHOD FOR MANUFACTURING TRANSGENIC PLANT PRODUCING IMMUNOGENIC COMPLEX PROTEINS AND IMMUNOGENIC COMPLEX PROTEINS OBTAINED THEREFROM

(71) Applicant: Chung-Ang University Industry Academic Cooperation Foundation, Seoul (KR)

(72) Inventors: Kisung Ko, Daejeon (KR); Deuk-su Kim, Seoul (KR); Kinarm Ko, Gyeonggi-do (KR)

(21) Appl. No.: 15/376,031

(22) Filed: Dec. 12, 2016

Related U.S. Application Data
(63) Continuation of application No. PCT/KR2015/005965, filed on Jun. 12, 2015.

(30) Foreign Application Priority Data
Jun. 12, 2014 (KR) 1020140071607

(51) Int. Cl. C12N 15/82; A61K 39/395; A61K 39/00; C07K 16/30; C07K 14/705

(52) U.S. Cl. CPC C12N 15/8258; C07K 16/3046; C07K 14/705; A61K 39/0011; A61K 39/39558; C07K 2319/30; C07K 2317/51; C07K 2317/515; C07K 2317/13

(57) ABSTRACT

The present invention relates to a method for manufacturing a transgenic plant producing immunogenic complex proteins and immunogenic complex proteins obtained therefrom and, more specifically, to a method for manufacturing a transgenic plant producing immunogenic complex proteins, a plant manufactured by the method, and immunogenic complex proteins obtained from the plant, wherein the method comprises the steps of: (a) manufacturing a transgenic plant expressing an antigen; (b) manufacturing a transgenic plant expressing an antibody specific to the antigen in step (a); and (c) cross-breeding the plants in steps (a) and (b) to manufacture a cross-bred plant. Immunogenic complex proteins can be mass-produced through the method for manufacturing a transgenic plant, comprising steps (a) to (c), and the transgenic plant manufactured by the method, of the present invention. Further, the immunogenic complex proteins (antigen-antibody complex) obtained from the plant have a gigantic four-dimensional structure, thereby having an excellent immune reaction boosting effect, thus exhibiting an excellent antibody producing capacity in a host animal, even without the use of an immune adjuvant.

### CROSS REFERENCE TO RELATED PATENT APPLICATIONS

This application is a continuation of and claims priority to PCT/KR2015/005965 filed Jun. 12, 2015, which is hereby incorporated herein by reference in its entirety and which claims priority from and the benefit of Korean Patent Application No. 10-2014-0071607 filed on 12 Jun. 2014, which is hereby incorporated herein by reference in its entirety.

### TECHNICAL FIELD

The present invention relates to a method for preparing a transformed (or transgenic) plant producing an immunogenic complex protein and an immunogenic complex protein obtained therefrom and, more specifically, to a method for preparing a transformed (or transgenic) plant producing an immunogenic complex protein, the method comprising: (a) preparing a transformed (or transgenic) plant expressing an antigen; (b) preparing a transformed (or transgenic) plant expressing an antibody specific to the antigen in step (a); and (c) mating the plants in steps (a) and (b) to prepare a mated plant, to a plant produced by the method, and to an immunogenic complex protein obtained from the plant.

### BACKGROUND ART

Vaccines are medicines that are used to induce immune responses against antigens for the purpose of defense against pathogenic infections, while vaccines that have recently been developed are mainly using recombinant proteins as antigens. Recombinant proteins have fewer side effects and are safer than killed vaccines or live attenuated vaccines, but due to their low immunogenicity thereof, an immune adjuvant is used together to induce sufficient immunity for the defense against infections. The immune adjuvant is a kind of vaccine additive that stimulates an immune response against a vaccine antigen to induce enhanced immunity while not having a specific antigen-antibody immune response itself, and its name is originated from “adjuvare” having a meaning of “help” or “enhance” in Latin.

Immune adjuvants are largely classified into three types depending on their mechanism: an antigen carrier; an immune enhancer; and a substance that stimulates an immune response and functions as a matrix against an antigen. The effective use of the immune adjuvant can obtain various effects of: (1) increasing immunogenicity of a recombinant antigen; (2) reducing the dose of antigen or the number of immunization; and (3) improving immunogenicity in infants and the elderly with weak immunity.

There is an aluminum salt, MF59, AS03, AS04 and the like as an immune adjuvant that is currently used for a vaccine under the approval of the US and the European Union. Aluminum salts developed as immune adjuvants of diphtheria toxoid vaccines in 1926 are currently the most widely used immune adjuvants, and have been almost exclusively used for human vaccines over the last 80 years. Aluminum salts are thought to be widely used for various vaccines and very safe, but are assumed to cause allergic reactions and have neurotoxicity. In addition, the aluminum salts strongly induce humoral immune responses mediated with antibodies, but hardly induce cellular immune responses and preclude the possibility of their cryopreservation.

As such, the immune adjuvants are used for vaccination, but the use thereof has been known to cause adverse effects (e.g., autism spectrum disorders (ASD) and allergy), and thus immune adjuvant-free vaccines are needed.

### PRIOR ART DOCUMENTS

### Patent Document

(Patent Document 1) Korean Patent No. 10-1054851

### Non-Patent Document

(Non-Patent Document 1) Zhe Lu, Kyung-Jin Lee, Yingxue Shao, Jeong-Hwan Lee, Yangkang So, Young-Kug Choo, Doo-Byoung Oh, Kyung-A Hwang, Seung Han Oh, Yeon Soo Han, and Ki sung Ko, Expression of GA733-Fc Fusion Protein as a Vaccine Candidate for Colorectal Cancer in Transgenic Plants, Journal of Biomedicine and Biotechnology. Volume 2012, Article ID 364240, 11 pages.

### DETAILED DESCRIPTION OF THE INVENTION

### Technical Problem

The present inventors, while researching on the production of immune adjuvant-free vaccines, verified that an antigen-antibody complex produced from a first-generation plant generated by cross-pollinating transformed (or transgenic) plants, which express an antigen and an antibody, respectively, triggers a hyperimmune response even without an immune adjuvant, and thus completed the present invention.

Therefore, an aspect of the present invention is to provide a method for preparing a transformed (or transgenic) plant producing an immunogenic complex protein, the method comprising:

(a) preparing a transformed (or transgenic) plant expressing an antigen;

(b) preparing a transformed (or transgenic) plant expressing an antibody specific to the antigen in step (a); and

(c) mating the plants in steps (a) and (b) to prepare a mated plant.

Another aspect of the present invention is to provide a plant preparing an immunogenic complex protein, the plant being produced by the method.

Still another aspect of the present invention is to provide an immunogenic complex protein derived from the plant.

Still another aspect of the present invention is to provide a vaccine composition comprising the immunogenic complex protein and a pharmaceutically acceptable carrier or diluent.

Still another aspect of the present invention is to provide a use of the immunogenic complex protein for preparing vaccine.

Still another aspect of the present invention is to provide an immunization method comprising administering an effective amount of the immunogenic complex protein to a subject in need thereof

### Technical Solution

In accordance with an aspect of the present invention, there is provided a method for preparing a transformed (or transgenic) plant producing an immunogenic complex protein, the method comprising:

(a) preparing a transformed (or transgenic) plant expressing an antigen;

(b) preparing a transformed (or transgenic) plant expressing an antibody specific to the antigen in step (a); and

(c) mating the plants in steps (a) and (b) to prepare a mated plant.

In accordance with another aspect of the present invention, there is provided a plant producing an immunogenic complex protein, the plant being produced by the method.

In accordance with still another aspect of the present invention, there is provided an immunogenic complex protein derived from the plant.

In accordance with still another aspect of the present invention, there is provided a vaccine composition comprising the immunogenic complex protein and a pharmaceutically acceptable carrier or diluent.

In accordance with still another aspect of the present invention, there is provided an use of immunogenic complex protein for preparing vaccine.

In accordance with still another aspect of the present invention, there is provided an immunization method comprising administering an effective amount of the immunogenic complex protein to a subject in need thereof

Hereinafter, the present invention will be described in detail.

The present invention provides a method for preparing a transformed (or transgenic) plant producing an immunogenic complex protein, the method comprising:

(a) preparing a transformed (or transgenic) plant expressing an antigen;

(b) preparing a transformed (or transgenic) plant expressing an antibody specific to the antigen in step (a); and

(c) mating the plants in steps (a) and (b) to prepare a mated plant.

In step (a), a transformed (or transgenic) plant expressing an antigen is prepared.

As used herein, the term “antigen” refers to any material that enters through contact with proper cells to induce a sensitive and/or immunoreactive state and responses with immune cells and/or antibodies of the sensitized object in vivo or in vitro in a verifiable manner. As used herein, the term “antigen” may be generally used with the same meaning as the term “immunogen”, and preferably refers to a molecule comprising at least one epitope capable of promoting a host immune system to trigger a secretory, humoral, and/or cellular immune response specific to the antigen. In addition, the term “antigenicity” or “immunogenicity” refers to the property of the antigen or immunogen, and means the property of triggering a secretory, humoral, and/or cellular immune response.

The term “immune response” is a self-defense system present in the animal body, and a biological phenomenon in which various materials or organisms invading from the outside are distinguished from the organism's own body and then these invaders are eliminated. Such a surveillance system for self-defense is largely composed of two mechanisms: one is a humoral immune mechanism and the other is a cellular immune mechanism. The humoral immune mechanism is made by antibodies existing in the serum, while the antibodies perform an important role of removing invading external antigenic materials by binding with the antigenic materials. Meanwhile, the cellular immune mechanism is made by several kinds of cells pertaining to the lymphatic system, and these cells are in charge of the function of directly disrupting invading cells or tissues. Therefore, the humoral immune mechanism is effective on external materials, such as bacteria, viruses, proteins, and composite carbohydrates, mainly existing outside the cells, whereas the cellular immune mechanism exerts its functions on various parasites, tissues, intracellular infection, and cancer cells. This double defense system is mainly performed by two kinds of lymphocytes, such as B cells or T cells. B cells produce antibodies, while T cells are involved in the cellular immune mechanism. These immune responses by B cells or T cells first respond to antigens invading the body, and constitute an immune system necessarily when the same kind of antigens continues to exist or repeatedly invade. Therefore, these immune responses are unique responses against specific antigens. Besides these antigen-specific immune responses, there is a kind of natural immune response in the body in which attacking cells are disrupted by a direct response even without exposure to a certain antigen. These immune responses are characterized in that neutrophils, macrophages, natural killer (NK) cells, and the like are involved in such immune responses to exert various functions regardless of the kind of target cells to be attacked.

The “epitope” refers to the simplest form of an antigenic determinant of a complicated antigenic molecule which is a specific part of an antigen recognized by an antibody or T cell receptor.

As used herein, the antigen according to the present invention includes, but is not limited to, polypeptides or proteins, non-protein molecules or fragments thereof. Preferably, the antigen of the present invention means a polypeptide or protein and a fragment thereof.

The antigen of the present invention may be an immunogenic material known in the art, and examples of the antigen include, but are not included to, a bacterial antigen or epitope, a fungal antigen or epitope, a plant antigen or epitope, a mold antigen or epitope, a viral antigen or epitope, a tumor (cancer) cell antigen or epitope, a toxin antigen or epitope, a chemical antigen or epitope, and a self-antigen or epitope.

The antigen of the present invention may preferably be a tumor-associated antigen. The kind of tumor-associated antigen is not limited as long as it is a tumor (or cancer)-associated antigen known in the art, but examples thereof include a breast cancer antigen, an ovarian cancer antigen, a prostate cancer antigen, a cervical cancer antigen, a pancreatic cancer antigen, a lung cancer antigen, a bladder cancer antigen, a colon cancer antigen, a testicular cancer antigen, a glioblastoma cancer antigen, an antigen associated with a B cell malignancy, an antigen associated with multiple myeloma, an antigen associated with non-Hodgkins lymphoma, an antigen associated with chronic lymphocytic leukemia, or a colorectal cancer antigen.

More specifically, the tumor-related antigen may be A33; ADAM-9; ALCAM; B1; BAGE; beta-catenin; CA125; carboxypeptidase M; CD5; CD19; CD20; CD22; CD23; CD25; CD27; CD28; CD32B; CD36; CD40; CD45; CD46; CD56; CD79a; CD79b; CD103; CD154; CDK4; CEA; CTLA4; cytokeratin 8; EGF-R; ephrin receptor; ErbB1; ErbB3; ErbB4; GAGE-1; GAGE-2; GD2; GD3; GM2; gplOO; HER-2/neu; human papillomavirus-E6; human papillomavirus-E7; integrin alpha-V-beta-6; JAM-3; KIDS; KID31; KSA(17-1A); LUCA-2; MAGE-1; MAGE-3; MART; MUC-1; MUM-1; N-acetylglucosaminyltransferase; oncostatin M (oncostatin receptor beta); p15; PIPA; PSA; PSMA; RAAG1O; ROR1; SART; sTn; TEST; TNF-α receptor; TNF-β receptor; TNF-γ receptor; transferrin receptor; VEGF receptor or GA733, but is not limited thereto.

The antigen of the present invention may preferably be a colorectal cancer cell surface protein, GA733. GA733 is an epithelial cell adhesion molecule (EpCAM; or also called 17-1A antigen, KSA, EGP40, GA733-2, ks 1-4, or esa). EpCAM is surface glycoprotein expressed by simple epithelial cells and tumorous cells derived therefrom. The EpCAM molecule is shown on the cellular surface from health tissues, but the expression thereof is upregulated in malignant tissues. EpCAM serves to adhere to epithelial cells in an oriented and highly ordered fashion (Litvinov, J Cell Biol. 1997, 139, 1337-1348).

The GA733 of the present invention may preferably be the polypeptide represented by SEQ ID NO: 1.

In addition, the “antigen” as used herein may further include an endoplasmic reticulum signal peptide (meaning the endoplasmic reticulum targeting sequence). The endoplasmic reticulum signal peptide (ER signal sequence) refers to an amino acid sequence that allows the recognition of a protein by signal recognition particles on the cytoplasmic reticulum to allow the protein to be translocated in the ER lumen. Herein, the kind and the amino acid sequence of the endoplasmic reticulum signal peptide are not limited as long as it is a plant endoplasmic reticulum signal peptide, and they may be referred to in literatures, such as US 20130295065 and WO 2009158716. Herein, the endoplasmic reticulum signal peptide may preferably be any one polypeptide selected from the group consisting of SEQ ID NO: 3, SEQ ID NO: 28, SEQ ID NO: 29, SEQ ID NO: 30, SEQ ID NO: 31, and SEQ ID NO: 32, and may most preferably be the polypeptide represented by SEQ ID NO: 3.

The endoplasmic reticulum signal peptide is characterized by being added (or linked) to the N-terminal of a protein which is intended to be expressed or synthesized in plant cells.

Herein, the antigen in step (a) may be preferably provided in a fusion form with an Fc antibody fragment. The term “fusion” is meant by encompassing all of chemical fusion and genetic fusion, and herein, preferably refers to genetic fusion. The “genetic fusion” means a linkage, which is composed of a linear covalent linkage formed through the genetic expression of the DNA sequence coding a protein.

Herein, the antigen provided in such a form is called a chimeric antigen. That is, the antigen of the present invention is preferably a chimeric antigen comprising (i) and (ii) below: (i) an immune response domain (IRD) comprising an antigenic protein; and (ii) a target binding domain (TBD) comprising an Fc antibody fragment.

The immune response domain (IRD) (i) refers to a portion of an antigenic protein, including the whole or a fragment thereof, which induces a substantial immune response, that is, the humoral and/or T cell reaction.

The antigenic protein refers to an antigenic material of a polypeptide or protein, and the antigen is described as above.

The target binding domain (TBD) (ii) includes at least one Fc antibody fragment-derived CH2 and CH3 domains, and refers to a portion capable of binding with an antigen-presenting cell (APC).

As used herein, the term “antibody” is used interchangeably with “an immunoglobulin (hereinafter, denoted by “Ig”), while it is a generic name of a protein that selectively acts on an antigen and is involved in the biological immune response. An antibody is composed of two pairs of light and heavy chains. These light and heavy chains of an antibody are polypeptides composed of several domains. In the whole antibody, each heavy chain includes a heavy chain variable region (VH) and a heavy chain constant region. The heavy chain constant region includes heavy chain constant domains CH1, CH2, and CH3 (antibody class: IgA, IgD, and IgG) and any heavy chain constant domain CH4 (antibody class; IgE and IgM). Each light chain includes a light chain variable domain (VL) and a light chain constant domain (CL). The structure of IgG antibody, which is one of naturally occurring whole antibodies, is for example shown in FIG. 2. The variable domains VH and VL may be subdivided into hypervariable regions called complementarity determining regions (CDRs) interspersed within regions that are more conserved, called framework regions (FR). Each VH and VL is composed of three CDRs and four FRs, arranged from amino-terminal to carboxy-terminal in the following order: FR1, CDR1, FR2, CDR2, FR3, CDR3, FR4 (Janeway, C. A., Jr. et al., (2001). Immunobiology., 5th ed., Garland Publishing; and Woof, J., Burton, D., Nat Rev Immunol 4 (2004) 89-99). The two pairs of heavy chain and light chain (HC/LC) can specifically bind to the same antigen. Thus, the whole antibody is a bivalent, monospecific antibody.

There are five types of mammalian antibody heavy chains denoted by the Greek letters: α, δ, ε, γ, and μ (Janeway, C. A., Jr., et al., (2001). Immunobiology., 5th ed., Garland Publishing). The class of antibody is defined by the type of its heavy chain; these chains are found in IgA, IgD, IgE, IgG, and IgM antibodies, respectively (Rhoades R. A., Pflanzer R G (2002). Human Physiology, 4th ed., Thomson Learning). Distinct heavy chains differ in size and composition; α and γ contain approximately 450 amino acids, while μ and ε have approximately 550 amino acids. Each heavy chain has two regions: the constant region and the variable region. The constant region is identical in all antibodies of the same isotype, but differs in antibodies of different isotype. Heavy chains γ, α and δ have a constant region composed of three constant domains CH1, CH2, and CH3 (in a line), and a hinge region for added flexibility (Woof, J., Burton, D., Nat Rev Immunol 4 (2004) 89-99); and heavy chains μ and ε have a constant region composed of four constant domains CH1, CH2, CH3, and CH4 (Janeway, C. A., Jr., et al., (2001). Immunobiology., 5th ed., Garland Publishing). The variable region of the heavy chain differs in antibodies produced by different B cells, but is the same for all antibodies that are produced by a single B cell or B cell clone. The variable region of each heavy chain is approximately 110 amino acids long and is composed of a single antibody domain.

In mammals, there are only two types of light chain, which are called lambda (λ) and kappa (κ). A light chain has two successive domains: one constant domain CL and one variable domain VL. The approximate length of a light chain is 211 to 217 amino acids.

In the present specification, unless otherwise particularly stated, IgG is understood to be a representative basic structure of an antibody.

The Fc fragment as used in the present invention may be derived from any one selected from the group consisting of IgG, IgA, IgD, IgE, and IgM, and preferably an IgG-derived Fc fragment. The IgG may be again divided into IgG1, IgG2, IgG3, and IgG4, while the Fc fragment of the present invention may most preferably be an IgG1-derived Fc fragment.

As used herein, the term “Fc fragment”, which is a segment obtained when an immunoglobulin (Ig) molecule is digested with papain, is a region of an immunoglobulin molecule excluding the variable region (VL) and the constant regions (CL) of the light chain and the variable region (VH) and the constant region 1 (CH1) of the heavy chain. That is, the Fc fragment means a dimer of two CH2-CH3 domain chains, while the two chains form a dimer structure by disulfide bonds. In addition, the Fc fragment may contain the whole or a part of the hinge region peptide in the heavy chain constant region. Also, the Fc fragment may be an extended Fc fragment that contains the whole or a part of the heavy chain constant region 1 (CH1) and/or the light chain constant region 1 (CL1) as long as it has substantially the same or an improved effect, compared with a natural one. Also, the Fc fragment may be a fragment having a deletion of a relatively long amino acid sequence corresponding to CH2 and/or CH3.

In addition, the Fc antibody fragment may be derived from the same species as a host (subject), to which a molecule or composition containing the chimeric antigen is to be administered, or one heterogenous to the host. For example, when the host is a human, the Fc antibody fragment may be derived from a human antibody, and the heterogenous Fc antibody fragment may be an Fc antibody fragment derived from a non-human mammal, for example, a cow, a goat, a swine, a mouse, a rabbit, a hamster, a rat, or a guinea pig.

The kind and amino acid sequence of the “Fc antibody fragment” of the present invention are not limited as long as it is an Fc antibody fragment known in the art. For example, the “Fc antibody fragment” of the present invention may be a polypeptide represented by SEQ ID NO: 4 (Fc fragment sequence of human IgG1) or a polypeptide obtained by adding a hinge region to said polypeptide sequence as represented by SEQ ID NO: 6.

As used herein, the term “antigen presenting cells (APC)” refers to auxiliary cells for an antigen-inducing event, which internalize antigens, process antigens, and mainly function by presenting antigenic epitopes to lymphocytes in the context of main tissue compatible complex (MHC) class I or II. The interaction between APCs and the antigen is a necessary step in the immune induction, for the lymphocytes are in contact with and recognize the contact, and thus can be activated. Examples of the APCs include macrophages, monocytes, Langerhans cells, dendrite cells interlocking with each other, follicular dendritic cells, and B cells.

The “target binding domain (TBD)” of the present invention contains at least one Fc antibody fragment-derived CH2 and CH3 domains, and thus can bind with the Fc receptor on APC. The Fc antibody fragment possesses an Fc receptor binding site, where it binds with the Fc receptor on APC.

The immune response domain (IRD) and the target binding domain (TBD) may be directly or indirectly linked to each other by genetic fusion means. Therefore, the chimeric antigen of the present invention includes the use of a linker molecule that connects TBD to IRD. Exemplary linker molecule includes a leucine zipper and biotin/avidin. For example, the other linker that can be used for the chimeric antigen is a peptide sequence. Such a peptide linker is about 2 to about 40 amino acids in length (e.g., about 4 to 10 amino acids). Exemplary peptide linker includes the amino acid sequence “SRPQGGGS”. The other linkers are known in the art, and are generally rich in glycine and/or alanine in consideration of the flexibility between regions connected by these linkers.

The chimeric antigens of the present invention may be monomeric (i.e., they contain a signal unit containing IRD and TBD), or may be multimeric (e.g., they contain a multiple unit containing IRD and TBD). The multimer may be, for example, a dimer, a trimer, a tetramer, a pentamer, a hexamer, a heptamer, or an octamer. In these multimers, the individual units may be the same with or different from each other, and some may be the same with each other and the others may be different from each other. The chimeric antigen of the present invention is preferably a dimer, while FIG. 1 shows a dimeric chimeric antigen of the present invention.

Also, the dimeric chimeric antigen may be referred to in U.S. Pat. No. 8,465,745; U.S. Pat. No. 8,029,803; and Korean Patent No. 10-1054851.

The chimeric antigen of the present invention preferably comprises:

(i) an immune response domain (IRD) comprising an antigenic protein; and

(ii) a target binding domain (TBD) comprising a hinge region, a CH2 domain, and a CH3 domain,

wherein the chimeric antigen is a dimer protein in which the C-terminal of the immune response domain is linked to the N-terminal of the target binding domain via a peptide linkage.

In addition, in step (a), the “antigen” may further contain an endoplasmic reticulum retention signal sequence (or ER retention signal peptide). The kind of the endoplasmic reticulum retention signal sequence is not limited as long as it is a plant endoplasmic reticulum retention signal sequence known in the art, and the details thereof may be referred to in WO 2009158716 or the following literature: Pagny et al., Signals and mechanisms for protein retention in the endoplasmic reticulum, Journal of Experimental Botany, Vol. 50, No. 331, pp. 157-64, February 1999.

The endoplasmic reticulum retention signal sequence of the present invention may preferably be KDEL (SEQ ID NO: 8), HDEL (SEQ ID NO: 23), and sequences in which one to five amino acids are added to the above sequence (e.g., SEKDEL of SEQ ID NO: 24, KHDEL of SEQ ID NO: 25, KEEL of SEQ ID NO: 26, and SEHDEL of SEQ ID NO: 27), and most preferably be KDEL represented by SEQ ID NO: 8.

KDEL may be exposed at a terminal of the amino acid sequence of the final product by inserting nucleotides encoding KDEL into a particular gene (herein, antigen-expressing gene). This induces the produced protein to exist within the endoplasmic reticulum in transformed cells without being released out of plant cells. The protein produced in host cells, to which the particular gene is introduced, is stored in the endoplasmic reticulum by the KDEL sequence, and undergoes the post-translation modification that may be implemented in a plant. This plays a key role in solving the problem due to the sugar structure difference between different species, which play an important role in the increase in the expression amount of an intracellular antibody protein and the immune response between different species. It is known that, in the antibody produced through endoplasmic reticulum (ER) retention procedure by the KDEL sequence, a high-mannose glycan structure is produced, and it is thought that the ER-type glycan chain in the glycoprotein (or also called oligomannose glycan-type) increases immune responses through a mannose receptor in dendritic cells or macrophages (Zhe Lu, et al., Expression of GA733-Fc Fusion Protein as a Vaccine Candidate for Colorectal Cancer in Transgenic Plants, Journal of Biomedicine and Biotechnology Volume 2012, Article ID 364240, 11 pages, doi: 10.1155/2012/364240).

The insert site of the endoplasmic reticulum retention signal sequence (KDEL) is not limited as long as it does not influence the immunogenicity or antibody binding ability of the antigen. In cases where the antigen of the present invention is provided in a form of a chimeric antigen as described above, the insert site of the endoplasmic reticulum retention signal sequence is not limited thereto, and may preferably be C-terminal region of the Fc antibody fragment.

The antigen in step (a) may be a GA733-FcK chimeric antigen represented by SEQ ID NO: 9. The GA733-FcK chimeric antigen is a dimeric protein in which a colorectal cancer cell surface protein, GA733, with an endoplasmic reticulum signal peptide linked thereto, a human IgG1 Fc fragment comprising a hinge region, and an endoplasmic reticulum retention signal sequence (designated by K) are connected to each other (see FIG. 1), and the details are referred to in Korean Patent No. 10-1054851 by the present inventors.

The term “transformation” means a modification of the genotype of a host cell due to the introduction of exogenous polynucleotides, and means an introduction of exogenous polynucleotides into a host cell regardless of a method for the transformation. The exogenous polynucleotides introduced into the host cell is incorporated into and maintained in the genome of the host cell, or is maintained without the incorporation thereinto, and the present invention includes both of them.

The term “introduction” means a manipulation of the artificial insertion of a gene or a gene group for its expression into a target cell or the addition of another gene (group) to the genome of the cell. The introduction of the gene may be performed by transduction via bacteriophage (bacteria), an indirect method via soil bacterium Agrobacterium spp., gene gun, electroporation, and microinjection, and the like, while a person skilled in the art can selectively use known gene introduction techniques according to target cells and the features of the inserted genes.

The transformation in step (a) means the introduction of polynucleotides encoding antigens (especially, antigenic proteins) into plant cells. The “preparing a transformed plant expressing an antigen” in step (a) may be performed by known methods of plant cell transformation, and for example, a desired gene is inserted into a vector to prepare a recombinant vector, and transforming the recombinant vector in a strain of the genus Agrobacterium, infecting the strain into plant cells.

The vector generally includes at least one of a signal sequence, a replication origin, at least one maker gene, enhance components, a promoter, and a transcription termination sequence, and preferably an expression vector. The expression vector is one type of vector that can express the selected polynucleotide. A single polynucleotide sequence is “operatively linked” to a regulatory sequence when the regulatory sequence influences the expression of the polynucleotide sequence (e.g., in terms of its level, timing, or expression loci). The regulatory sequence is a sequence that affects the expression (e.g., in terms of level, timing, or location of expression) of the nucleic acid to which it is operatively linked. The regulatory sequence, for example, can exert its effects directly on the regulated nucleic acid, or through the action of one or more other molecules (e.g., polypeptides that bind to the regulatory sequence and/or the nucleic acid). The regulatory sequences include promoters, enhancers, and other expression control elements.

Standard recombinant DNA and molecular cloning techniques, as molecular biological technology known in the art, are described in the following literatures. (Sambrook, J., Fritsch, E. F. and Maniatis, T., Molecular Cloning: A Laboratory Manual, 2nd ed., Cold Spring Harbor Laboratory: Cold Spring Harbor, N.Y., 1989; by Silhavy, T. J., Bennan, M. L. and Enquist, L. W., Experiments with Gene Fusions, Cold Spring Harbor Laboratory: Cold Spring Harbor, N.Y., 1984; and by Ausubel, F. M. et al., Current Protocols in Molecular Biology, published by Greene Publishing Assoc. and Wiley-Interscience, 1987).

Following the safe transformation, the transformed (or transgenic) plant is propagated. The propagation means an increase in the number of plants. A method for the propagation of the plant is not limited as long as the characteristics of the reproduced plant and the expression characteristics of a mother gene-grafted plant, and may preferably be micro-proliferation.

The micro-proliferation is a method of growing a second-generation plant from a single tissue sample cut from a selected mother plant or a cultivated species. This method allows the mass-production of plants having a preferable tissue and expressing a target protein. The newly generated plant has the same genetic constitution as the original plant and has all the same features as the original plant. The micro-proliferation enables mass-production of excellent plant material for a short period of time, and enables a prompt proliferation of selected crops while conserving features of originally gene-grafted or transformed (or transgenic) plants. The plant cloning method has advantages of prompt plant proliferation and excellent and uniform plants.

In step (b), a transformed (or transgenic) plant expressing an antibody specific to the antigen in step (a) is prepared.

The “antibody” has been described as above.

The term “specific” refers to a state in which one of the specifically bound molecules never shows a significant bond to a molecule except for one or a plurality of counter molecules. Herein, the term means specificity with which an antibody can bind to only one antigen, and is also used for a case in which an antigen binding domain is specific to a particular epitope among a plurality of epitopes contained in a certain antigen. Also, in cases where the epitope bound to the antigen binding domain is included in a plurality of different antigens, an antigen binding molecule having the corresponding antigen binding domain may bind with various antigens containing the corresponding epitope.

Herein, the “antibody specific to the antigen in step (a)” may be any type selected from the group consisting of IgG, IgA, IgD, IgE, and IgM, and may be provided in a form of a whole antibody that is derived from the nature. In addition, the antibody specific to the antigen in step (a) includes a monoclonal antibody and a polyclonal antibody, and preferably a monoclonal antibody.

As used herein, the term “monoclonal antibody” refers to a protein molecule that is directed by a single antigenic region (single epitope) and specifically binds thereto. The monoclonal antibody substantially represents an antibody obtained from a group of homogenous antibodies. In other words, individual antibodies constituting said group are identical excluding mutants that may be present naturally in small quantity. The monoclonal antibody may be prepared by known methods in the art of preparing monoclonal antibody, for example, but is not limited to, a hybridoma method first described in the literature (see, Kohler et al., (1975) Nature 256:495), or a recombinant DNA method (see, U.S. Pat. No. 4,816,567). The monoclonal antibody may also be isolated from phage antibody libraries using the techniques described in the literatures (see, Clackson et al. (1991) Nature 352: 624-628, Marks et al. (1991) J. Mol. Biol. 222: 581-597, and Presta (2005) J. Allergy Clin. Immunol. 116:731).

As used herein, the term “polyclonal antibody” means an antibody mixture containing two or more monoclonal antibodies, and may respond to a plurality of epitopes.

In addition, the “antibody specific to the antigen in step (a)” includes all types of multivalent antibodies, but may preferably be a bivalent antibody. The bivalent antibody has a structure of a two-armed antibody having two identical ABSs, which is shown in FIG. 2.

The “multivalent” antibody is an antibody comprising two or more antigen-binding sites. The multivalent antibody includes bivalent, trivalent, tetravalent, pentavalent, hexavalent, heptavalent, or higher binding valent antibodies.

When a chimeric antigen is used in step (a), the same kind of antibody derived from the Fc fragment contained in the chimeric antigen is preferably used as the antibody in step (b). For example, when the chimeric antigen containing an IgG-derived Fc fragment is used in step (a), the antibody in step (b) is IgG specific to the chimeric antigen in step (a).

The antibody in step (b) may be derived from the same species as a host (subject), to which a molecule or composition containing the chimeric antigen in step (a) is to be administered, or one heterogenous to the host. For example, when the host is a human, the antibody may be derived from a human, while the heterogenous antibody may be an antibody derived from a non-human mammal, for example, a cow, a goat, a swine, a mouse, a rabbit, a hamster, a rat, or a guinea pig.

In step (b) of the present invention, the antibody may further include an endoplasmic reticulum retention signal sequence (KDEL). The endoplasmic reticulum retention signal sequence is described as above, and the insertion site is limited as long as it does not influence the antigen recognition and the binding ability of an antibody, but the insertion site may preferably be a terminal of the antibody protein peptide sequence, more preferably the C-terminal of the antibody protein peptide sequence.

The antibody in step (b) of the present invention is characterized by being a bivalent antibody (dimeric protein) specific to the GA733-FcK chimeric antigen, the antibody being represented by SEQ ID NO: 11 (heavy chain) and SEQ ID NO: 13 (light chain). The antibody specific to the GA733-FcK chimeric antigen is an antibody to GA733 protein, which is substantially an antigenic site, and named CO17-1A. Herein, the antibody in step (b) is, preferably, a bivalent antibody represented by: SEQ ID NO. 12 (heavy chain) comprising an endoplasmic reticulum retention sequence at the C-terminal of the heavy chain of SEQ ID NO: 11 and SEQ ID NO: 13 (light chain), and herein, named as CO17-1AK (see FIG. 2).

The transformation in step (b) means the introduction of polynucleotides encoding the antibody into a plant cell, and the “transformation” and the proliferation of transformed plants have been described above.

In step (c), the plants in steps (a) and (b) are mated to prepare a mated plant.

The term “mating” refers to a procedure in which, for the purpose of sexual reproduction, two individuals with opposite sexes or different mating types are fertilized by various methods, and male and female gametes fuse together to create a zygote, and here, the sameness or difference between the genotypes of both parents does not matter. The mating of two individuals with different genotypes is called “crossing”, and the “mating” of the present invention includes the crossing.

The mating of the present invention may be performed by known mating or crossing methods. The mating may be performed by, for example, but is not limited to, cross-pollination.

The species of the “plant” in steps (a) to (c) includes all of a case were the plants used in steps (a) and (b) are homogenous species and the plant in step (c) obtained by mating the plants is also a homogenous species, and a case where the plants used in steps (a) and (b) are heterogenous species and the plant in step (c) obtained by mating the plants is also a heterogenous species (especially, hybrid). Preferably, the plants used in steps (a) and (b) may be homogenous species and the plant in step (c) obtained by mating the plants may also be a homogenous species.

The “plant” in steps (a) to (c) is not limited as long as it may be a plant into which an exogenous gene may be introduced, but may be, for example, monocotyledonous plants, including rice, wheat, barley, bamboo shoot, corn, taro, asparagus, onion, garlic, green onion, leeks, wild chive, Chinese yam, and ginger. Examples of the dicotyledonous plant may include, but are not limited to, Arabidopsis thaliana, eggplant, Nicotiana tabacum, red pepper, tomato, burdock, crown daisy, lettuce, balloon flower, spinach, beet, sweet potato, celery, carrot, water parsley, parsley, Chinese cabbage, cabbage, mustard root, watermelon, oriental melon, cucumber, pumpkin, strawberry, soybean, mung beans, kidney beans, birds-foot trefoil, potato, duckweed, perilla seeds, dove beans, and pea. Preferably, the dicotyledonous plant may be Nicotiana tabacum.

In the mated plant prepared in step (c), heterogenous proteins produced from the parent-generation plants (that is, the plants in steps (a) and (b)) may be simultaneously expressed, and a new type of fusion protein in which all or some domains of the heterogenous proteins are fused may be produced. Specifically, the new type of fusion protein produced in the present invention means a protein in which some domains, respectively, from the chimeric antigen in step (a) and the antibody in step (b) are fused, while an example thereof is shown in FIG. 10c.

The fusion protein having a structure in FIG. 10c is called “Fab arm exchanged fusion protein”, and specifically means a fusion protein having a structure comprising:

(i) an antigenic protein;

(ii) a Fab antibody fragment specific to the antigenic protein in (i) above; and

(iii) an Fc antibody fragment.

The term “antigenic protein” and “Fc antibody fragment” have been described as above.

As used herein, the term “Fab antibody fragment (or arm)” refers to an antibody fragment composed of CH1 domain (first constant domain) and a variable region of each of one light chain and one heavy chain. That is, the Fab antibody fragment is a fragment that includes VH and CH1 domains of the heavy chain and VL and CL domains of the light chain, and exhibits single specificity to an antigen. The digestion of the antibody with papain generates two identical antigen binding fragments, called “Fab” fragments, each having a single antigen-binding site, and the remaining “Fc” fragment.

As used herein, the term “Fab arm exchange” refers to the swapping of an antibody half-molecule having one Fab fragment (that is, one heavy chain and light chain attached thereto).

The structure of the “Fab arm exchanged fusion protein” of the present invention is characterized in that, on the basis of the Fc antibody fragment as a symmetric axis, the CH2 and CH3 domains at one side of the Fc antibody fragment (iii) are linked with the antigenic protein (i), while the CH2 and CH3 domains at the other side are linked to the Fab fragment (ii) (see FIG. 10c).

Here, the CH2 and CH3 domains at one side of the Fc antibody fragment (iii) and the antigenic protein (i) are derived from the chimeric antigen in step (a), while the CH2 and CH3 domains at the other side of the Fc antibody fragment (iii) and the Fab fragment (ii) are derived from the antibody in step (b).

Preferably, the Fab arm exchanged fusion protein of the present invention is prepared by fusing some domains out of the GA733-FcK chimeric antigen and the antibody specific to the GA733-FcK chimeric antigen (i.e., CO17-1AK), and specifically, the Fab arm exchanged fusion protein may be a fusion protein:

(i) colorectal cancer cell surface protein GA733;

(ii) IgG Fab fragment specific to the GA733 protein (i) above; and

(iii) an Fc antibody fragment.

Here, the CH2 and CH3 domains at one side of the IgG Fc fragment (iii) and GA733 (i) are derived from the GA733-FcK chimeric antigen in step (a), while the CH2 and CH3 domains at the other side of the Fc antibody fragment (iii) and the Fab fragment (ii) are derived from the antibody specific to the GA733-FcK chimeric antigen (i.e., CO17-1AK) in step (b).

The mated plant prepared in step (c) is characterized in that the immunogenic complex protein is expressed in the plant cell.

As used herein, the term “immunogenic complex protein” refers to an antigen-antibody complex obtained by binding an epitope of an antigen to an antigen binding site of an antibody. Specifically, the term means a protein complex obtained by binding an epitope region (hereinafter, referred to as “antigenic site”) of the chimeric antigen protein in step (a) to the antigen binding site (ABS) of the antibody in step (b). The “binding an epitope site of an antigenic protein to an antigen binding site of an antibody” is known in the art, and may be obtained by, preferably, a non-covalent bond.

As such, the immunogenic complex protein of the present invention is obtained from the binding only between an epitope of an antigenic site and an antigen binding site (ABS) of an antibody, and is differentiated from the meaning of the foregoing fusion.

With respect to the combination of two of the chimeric antigen in step (a) and the antibody in step (b), the specific forms of the antigen-antibody complex are, but are not limited thereto, for example, a chimeric antigen-antibody monomolecular form in which one chimeric antigen and one antibody are bound (shown in FIG. 10a); a linear form in which the linkage between chimeric antigens is mediated by antibodies functioning as a bridge role (i.e., cross-linkage of chimeric antigens and antibodies, shown in FIG. 10b); and a polymeric structure in which the chimeric antigen-antibody monomolecular monomers are polymerized (e.g., a pentameric form of the chimeric antigen-antibody monomolecular monomers, shown in FIGS. 11a and 11b).

In addition, the immunogenic complex protein of the present invention may include the foregoing Fab arm exchanged fusion protein. For example, two of the Fab arm exchanged fusion proteins may be bound (shown in FIG. 10d), or two or more of only the Fab arm exchanged fusion proteins are bound in a linear form (shown in FIG. 10e). Alternatively, as shown in FIG. 10f, the foregoing chimeric antigens, antibodies specific thereto, and Fab arm exchanged fusion proteins may be bound in a linear form. Here, the structural diversity of the immunogenic complex protein according to the present invention is due to the structure feature in which the Fab arm exchanged fusion protein has an antigen and an antigen binding site specific to the antigen simultaneously.

The combinations of several immunogenic complex proteins have a quaternary structure of a large protein as shown in FIGS. 10 to 11.

The structure of a protein is defined by the primary, secondary, tertiary, and quaternary structures. The primary structure represents the information of amino acid sequence constituting a protein. The secondary structure represents a helix, strand, or random coil, which is a specific pattern configured by congregating amino acid residues. In addition, the tertiary structure represents a three-dimensional structure by congregating the secondary structures. The quaternary structure represents a form in which some protein chains are congregated to interact with each other.

Therefore, the method of the present invention comprising the steps (a) to (c) has an excellent effect in that the immunogenic complex proteins prepared by the method form strong bonds, and as described above, and enter dendritic cells, ultimately similar to opsonization through a scheme for forming a large quaternary molecular structure from a linear form or circular form, and thus constructing a vaccine structure for allowing efficient antigen presenting in plants.

This is well described in the following examples of the invention.

<Example 4> verified that the immunogenic complex protein of the present invention was generated in a pentameric structure of chimeric antigen-antibody monomolecules similar to IgM, as shown in FIG. 11. <Example 5> verified that the immunogenic complex protein according to the present invention had an excellent vaccine effect.

Therefore, the present invention provides a plant for producing the immunogenic complex protein produced by the method comprising steps (a) to (c).

The immunogenic complex protein is described as above, and specifically, the immunogenic complex protein may be a chimeric antigen-antibody complex of GA733-FcK chimeric antigen and an antibody specific thereto. The combinations (i.e., combinations of immunogenic complex protein) and morphology (structure) of the antigen-antibody complex are described as above.

In addition, the present invention provides an immunogenic complex protein derived from the plant.

The immunogenic complex protein is obtained from the plant prepared through steps (a) to (c) above.

The “obtaining the protein from the plant” may be performed by known methods for obtaining a plant cell-derived protein, and an example of the method may include, but is not limited to, a method for disrupting and pulverizing a mated plant and homogenizing the same in an extraction buffer. The extraction buffer may be a known plant protein extraction buffer, and examples thereof may include, but are not limited to, phosphate buffered saline (PBS), or a composition containing tris-HCl pH 8, dithiothreitol (DTT), protease inhibitor (e.g., aprotinin, pepstatin, leupeptine, phenyl methyl sulphonyl fluoride, and [(N—(N-(L-3-trans-carboxy oxirane-2-carbonyl)-L-leucyl)-agmantine].

A protein purification procedure may be further included herein. The “protein purification” may be performed by ordinary methods, and for example, salting out (e.g., ammonium sulfate precipitation, sodium phosphate precipitation), solvent precipitation (e.g., protein fraction precipitation using acetone, ethanol, etc.), dialysis, gel filtration, ion exchange, column chromatography, such as reverse-phase column chromatography, and ultrafiltration may be employed alone or in combination (Deutscher, M., Guide to Protein Purification Methods Enzymology, vol. 182. AcademicPress. Inc., San Diego, Calif. (1990)). Only the immunogenic complex protein of the present invention (i.e., a large quaternary structure antigen-antibody complex) can be obtained at a high concentration by the protein purification procedure.

Thus, the immunogenic complex protein of the present invention may be prepared by a method comprising the following steps.

(a) preparing a transformed (or transgenic) plant expressing an antigen;

(b) preparing a transformed (or transgenic) plant expressing an antibody specific to the antigen in step (a);

(c) mating the plant in step (a) and the plant in step (b) to prepare a mated plant; and

(d) obtaining a protein component from the mated plant.

Also, the method may further include

(e) purifying the protein obtained in step (d).

The combination and morphology (structure) of the immunogenic complex protein of the present invention are described as above (see FIGS. 10 and 11), and includes all of the linear form or circular form. Preferably, the immunogenic complex protein may be in a circular form.

The immunogenic complex protein of the present invention, as shown in FIGS. 10 and 11, is characterized by having a large quaternary structure. The immunogenic complex protein, when having a linear form, may be larger than a protein existing as a monomer, and is in a pre-stage for forming a circular form and may be smaller than the circular form. The immunogenic complex protein of the present invention, when having a circular form, may have a diameter of preferably 10-50 nm, and more preferably 20-30 nm, most preferably for antigen presenting.

The immunogenic complex protein of the present invention, as shown in FIGS. 10 and 11, has a large quaternary structure, thereby exhibiting an excellent effect in boosting immune response. Particularly, the immunogenic complex protein of the present invention has an excellent antibody producing ability in host animals even without using an immune adjuvant which is known to cause an adverse effect. In addition, compared with the existing antigen-antibody binding generated when an antigen and an antibody are placed at the same point in vitro in the prior art, the antigen-antibody complex generated from the plant mating of the present invention has tighter binding, leading to an excellent immunopotentiating effect.

Therefore, the present invention provides a vaccine composition comprising the immunogenic complex protein.

Also, the present invention provides a use of the immunogenic complex protein for preparing a vaccine.

As used herein, the term “vaccine” or “vaccine composition” refers to a composition that stimulates an immune response, and herein, the term is used interchangeably with an immunogenic composition as the same meaning. The vaccine includes both a prophylactic vaccine and a therapeutic vaccine. The prophylactic vaccine, in order to internalize a greater immune response when a subject is exposed to an antigen, induces an immune response before the exposure to a material containing the antigen, and thus increases the ability to resist a material or cell transferring the antigen. The therapeutic vaccine is used in a manner of being administered to a subject already having a disease relevant to an antigen in relation to the vaccine, and provides an increased ability to fight a disease or cell transferring the antigen, and thus increases the immune response of the subject against the antigen.

The vaccine composition is characterized by containing the immunogenic complex protein of the present invention. Therefore, the target disease targeted by the vaccine composition is determined by a substantial immune reaction domain contained in the immunogenic complex protein. For example, when the immune response domain is a tumor-related antigen, the vaccine composition of the present invention is administered to prevent and treat a corresponding tumor disease.

The vaccine composition of the present invention may be administered alone or in combination with a known compound which is effective in preventing and treating a target disease.

The vaccine composition of the present invention may be administered to mammals including humans by any method. For example, the vaccine composition of the present invention may be administered orally or parenterally. The parenteral administration may be, but is not limited to, intravenous, intramuscular, intra-arterial, intramedullary, intradural, intracardiac, transdermal, subcutaneous, intraperitoneal, intranasal, intestinal, topical, sublingual, or rectal administration.

The vaccine composition of the present invention is characterized by containing the immunogenic complex protein, and may further contain a pharmaceutically acceptable carrier, excipient, or diluent. As used herein, the term “pharmaceutically acceptable” refers to non-toxic which means to be physiologically acceptable, not to inhibit the action of an active ingredient when administered to humans, and not to normally cause an allergic reaction or similar reactions, such as gastroenteric troubles and dizziness.

The term “carrier” refers to a material that facilitates the addition of compounds into cells or tissues. Examples of the pharmaceutically acceptable carrier may further include a carrier for oral administration or a carrier for parenteral administration. The carrier for oral administration may include lactose, starch, cellulose derivative, magnesium stearate, stearic acid, and the like. In addition, the carrier for oral administration may include various drug delivery materials used for oral administration of peptide preparations. Also, the carrier for parenteral administration may include water, suitable oil, saline, aqueous glucose, and glycol, and may further include a stabilizer and a preservative. Suitable examples of the stabilizer include an antioxidant, such as sodium hydrogen sulfite, sodium sulfite, or ascorbic acid. Suitable examples of the preservative include benzalkonium chloride, methyl- or propyl-paraben, and chlorobutanol. The pharmaceutical composition of the present invention may further contain, in addition to the above ingredients, a lubricant, a wetting agent, a sweetening agent, a flavoring agent, an emulsifier, a suspending agent, and the like. Other pharmaceutically acceptable carriers and preparations may be referred to in the literature (Remington's Pharmaceutical Sciences, 19th ed., Mack Publishing Company, Easton, Pa., 1995).

Further, the present invention provides an immunization method characterized by administering an effective amount of the immunogenic complex protein to a subject in need thereof.

The term “subject” refers to an animal, preferably a mammal, and especially, an animal including a human being, and may be cells, tissues, organs, or the like, derived from the animal. The subject may be a patient in need of treatment.

The term “immunization” refers to the induction of a secretory, humoral, and/or cellular immune response against the immunogenic complex protein in a subject when the immunogenic complex protein according to the present invention is administered to the subject, where such an immunization leads to a prophylactic or therapeutic effect on a target disease.

The target disease is determined by an antigen contained in the immunogenic complex protein according to the present invention, that is, a substantial immune response domain. As the target disease, any disease may be included as long as a disease is known to be caused by a specific antigen, that is, such a disease-causing antigen is contained in the immunogenic complex protein of the present invention and thus can be favorably used for its prevention or treatment. The kind of target disease is not limited, but examples thereof may include tumor diseases, autoimmune diseases, metastatic diseases, degenerative diseases, viral or bacterial infections, prion disease, motor neuron disease (MND), and the like.

Specific examples of the target disease may include melanoma, adenocarcinoma, lung cancer, non-small cell lung cancer, ovarian cancer, cervical cancer, prostate cancer, bladder cancer, colon cancer, colorectal cancer, testicular cancer, B cell malignant tumor, multiple myeloma, non-Hodgkin's lymphoma, chronic lymphocytic leukemia, muscle cancer, pancreatic cancer, brain tumor, astroblastoma, glioblastoma, breast cancer, chordoma, allergy, asthma, multiple sclerosis (MS), diabetes, rheumatoid arthritis, urinary incontinence, osteoporosis, Alzheimer's disease, Lewy body disorder (LBD) caused by synuclein protein abnormality, degenerative neural diseases such as Parkinson's disease (PD) and multiple system atrophy (MSA), acquired immune deficiency syndrome (AIDS), hepatitis caused by hepatitis B or C virus, infections by human papilloma virus (HPV) and tumors caused therefrom, infections by Chlamydia pneumonia, infections by Escherichia coli, stomach ulcer caused by Helicobacter pylori, malaria, tuberculosis, infections by Candida such as Candida albicans, anthrax, sepsis, variant Creutzfeldt-Jakob disease (vCJD), scrapie, and amyotropic lateral sclerosis (ALS).

Preferably, the target disease of the immunogenic complex protein according to the present invention may be a tumor disease, and more preferably, a colorectal cancer or colon cancer.

The term “effective amount” refers to an amount that exhibits a prophylactic (or preventative) or therapeutic effect of the immunogenic complex protein of the present invention on the target disease, and an amount sufficient to induce a secretory, humoral, and/or cellular immune response against the immunogenic complex protein of the present invention in a subject administered with the immunogenic complex protein.

A total effective amount of the protein of the present invention may be administered to a subject in a single dose, or may be administered in a multiple dose by the fractionated treatment protocol for the long-period administration. Also, the contents of active ingredients may vary depending on the purpose of administration. The effective dose for each subject may be determined considering various factors, such as the age, body weight, health condition, sex, disease severity, diet, and excretion rate of the subject in need of administration, as well as the type and severity of the target disease, route of administration, and frequency of administration. Therefore, a person having ordinary skill in the art could determine an appropriate effective dose according to the purpose of administration. The effective dose may be determined by monitoring the therapeutic efficacy using an assay method of determining the activity of immune cells after the protein according to the present invention is administered or in vivo assay that is widely known. The pharmaceutical composition of the present invention is not particularly limited to the dosage form, route of administration, and administration method as long as the effect of the present invention can be exhibited.

The route of administration of the immunogenic complex protein of the present invention has been described as above. The immunogenic complex protein of the present invention may be administered together with a pharmaceutically acceptable carrier, an excipient, or a diluent. The carrier, excipient, or diluent has been described as above.

The immunogenic complex protein according to the present invention may be administered alone or in combination with a known compound having an effect of preventing and treating a target disease.

### Advantageous Effects

Through the method for preparing a transformed (or transgenic) plant, comprising steps (a) to (c), and the transformed (or transgenic) plant prepared by the method, immunogenic complex proteins can be mass-produced safely and economically. The immunogenic complex protein (antigen-antibody complex) obtained from the plant has a large quaternary structure, thereby having an excellent effect in boosting immune response, and thus exhibits an antibody producing ability in a host animal even without using an immune adjuvant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts chimeric antigen, and specifically, illustrates a structure of colorectal cancer cell surface specific protein-Fc (GA733-FcK).

FIG. 2 depicts bivalent, monospecific antibody against antigen, and specifically, illustrates a structure of colorectal cancer cell surface specific protein-Fc-specific antibody (CO17-1AK)

FIG. 3 is a schematic diagram of a procedure obtaining a T1-generation plant through cross-pollination of plants expressing colorectal cancer cell surface specific protein-Fc (GA733-FcK) and colorectal cancer cell surface specific protein-Fc-specific antibody (CO17-1AK), respectively.

FIG. 4 illustrates results of selecting plants having two genes (GA733-FcK and CO17-1AK) out of T1-generation plants (NOs. 1 to 13) using PCR (GA: standard GA733-FcK, CO: standard mAb CO17-1AK, NT: Non-Transgenic plant, HC: heavy chain of CO17-1AK, LC: light chain of CO17-1AK).

FIGS. 5A and 5B illustrate western blot results of expression of GA733-FcK gene (FIG. 5A) and CO17-1AK gene (FIG. 5B) in plant NOs 3, 4, 6, 9, and 11.

FIG. 6 illustrates the results of investigating, using SDS-PAGE, whether two proteins (GA733-FcK and CO17-1AK) were purified in T1-generation plant NO. 4.

FIG. 7 illustrates the results investigating, using two-color western blot, whether two proteins (GA733-FcK and CO17-1AK) were simultaneously expressed in protein samples purified from T1-generation plant NO. 4.

FIG. 8a is a schematic diagram showing the binding of capture antibody and antigen (chimeric antigen in the present invention, specifically, GA733-FcK protein) and the binding type of detection antibody recognizing the binding antigen-antibody complex, in sandwich ELISA (capture antibody: green, detection antibody: blue).

FIG. 8b illustrates the comparative results of binding signals of capture antibody and protein, in sandwich ELISA, when different protein samples (GA^{P}, GA^{P}+CO^{P}, GA^{P}×CO^{P}) were treated on the same capture antibody (CO^{M} or CO^{P}).

FIG. 9a illustrates the SPR measurement results when GA^{P}-fixed chip was treated with CO^{M}, CO^{P}, GA^{P}+CO^{P}, and GA^{P}×CO^{P} samples.

FIG. 9b illustrates the SPR measurement results when CO^{P}-fixed chip was treated with GA^{M}, GA^{P}, GA^{P}+CO^{P}, and GA^{P}×CO^{P} samples.

FIGS. 10a-10f exemplify complex structures showing a linear form, out of immunogenic complex proteins expressed in the T1-generation plant of the present invention. Specifically:

FIG. 10a shows the simplest type of chimeric antigen-antibody dimeric form, out of chimeric antigen-antibody complexes expressed in T1-generation plant.

FIG. 10b shows an example of a linear form of chimeric antigen-antibody complex, out of chimeric antigen-antibody complexes expressed in T1-generation plant.

FIG. 10c shows, as an example of a fusion protein expressed in T1-generation plant, a structure of a fusion protein called “Fab arm exchanged fusion protein” herein.

FIG. 10d shows a protein dimeric form by the “Fab arm exchanged fusion protein”.

FIG. 10e shows an example of a linear form of complex, out of protein complexes by the “Fab arm exchanged fusion protein”.

FIG. 10f shows another example of a linear form of complex, out of protein complexes by the “Fab arm exchanged fusion protein”.

FIGS. 11a and 11b exemplify complex structures showing a circular form, out of immunogenic complex proteins expressed in the T1-generation plant of the present invention. Specifically:

FIG. 11a shows an example of a pentamer structure, out of circular polymerization types of chimeric antigen-antibody monomolecules expressed in T1-generation plant.

FIG. 11b shows another example of a pentamer structure, out of circular polymerization types of chimeric antigen-antibody monomolecules expressed in T1-generation plant.

FIG. 12 illustrates electron microscopic observation images of a structure of a protein sample obtained in the parent-generation plant transformed to express GA733-FcK (chimeric antigen). On each image, scale bar expressed by a white horizontal bar indicates 10 nm.

FIG. 13 illustrates electron microscopic observation images of a structure of a protein sample obtained in T1-generation plant. On each image, scale bar expressed by a white horizontal bar indicates 10 nm.

FIG. 14 illustrates SPR confirmation results of vaccination effect (antibody production effect in serum) after each protein sample was injected into mice without an immune adjuvant.

FIG. 15 illustrates the confirmation results of interleukin-4 (IL-4) production in mice vaccinated with respective proteins.

FIG. 16 illustrates the confirmation results of interleukin-10 (IL-10) production in mice vaccinated with respective proteins.

FIG. 17 illustrates the confirmation of activity of anti-colorectal cancer antibody in serum obtained from mice administered with respective vaccine candidate materials, showing the size of colorectal cancer over time.

FIG. 18 illustrates mass analysis results of the sugar structure obtained by purifying GA^{P}(GA733-FcK), CO^{P}(CO17-1AK), and GA^{P}×CO^{P} (GA733-FcK× CO17-1AK) in plant, confirming that GA^{P}×CO^{P} obtained through cross-pollination had the similar sugar structure pattern to its parent-generation plant.

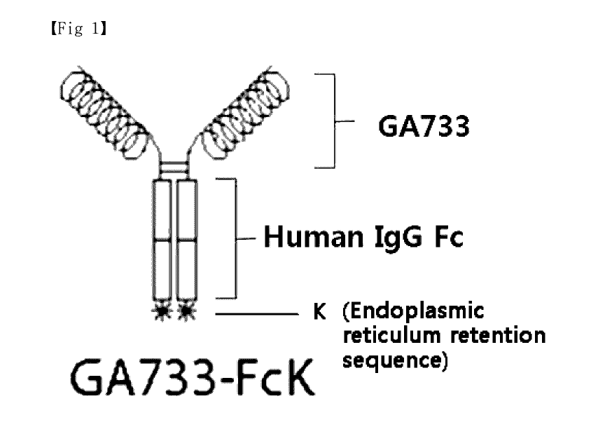

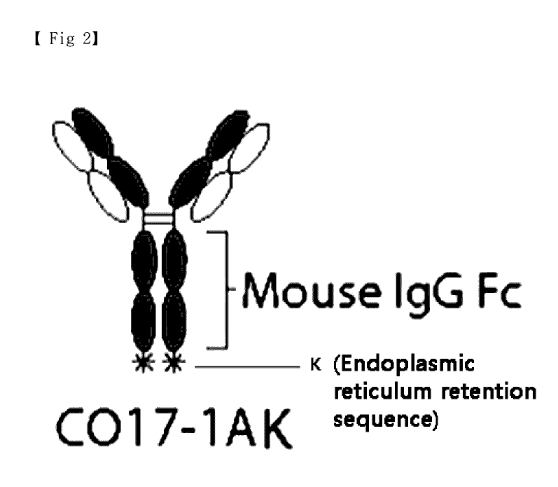

### MODE FOR CARRYING OUT THE INVENTION

Hereinafter, the present invention will be described in detail.

However, the following examples are merely for illustrating the present invention and are not intended to limit the scope of the present invention.

### Example 1

Preparation of Antigen-Expressing Transformed (or Transgenic) Plant and Antibody-Expressing Transformed (or Transgenic) Plant

Colorectal cancer cell surface specific protein-Fc (GA733-FcK antigen) was prepared by the same method as described in Korean Patent NO. 10-1054851 by the present inventors, and the literature by Zhe Lu et al.

Briefly speaking, the genes encoding the colorectal cancer cell surface specific protein GA733 (SEQ ID NO: 1) modified by N-terminal extension with a 30-aa plant ER signal peptide (SEQ ID NO: 3) and the human IgG1 Fc sequence (SEQ ID NO: 6) with ER retention signal (SEQ ID NO: 8) added at the IgG Fc C-terminal were disposed to arrange a gene sequence (see SEQ ID NO: 10) to express GA733-FcK recombinant fusion protein (SEQ ID NO: 9). An expression cassette was constructed by disposing a cauliflower mosaic virus (CaMV) 35S promoter and a tobacco etch viral 5-leader sequence (TEV) in front of the GA733-FcK gene. The constructed colorectal cancer cell surface specific protein-Fc expression cassette as such was inserted into the pBINPlus vector using restriction enzyme HindIII to prepare a plant expression vector.

In order to express, in a plant, mAb CO17-1A (heavy chain: SEQ ID NO: 11, light chain: SEQ ID NO: 13), known as an antibody against the colorectal cancer cell surface specific protein (GA733), the gene sequence of the ER retention signal was added to the C-terminal of the IgG heavy chain of the mAb CO17-1A, which was named mAb CO17-1AK (heavy chain: SEQ ID NO: 12, light chain: SEQ ID NO: 13). The gene sequences encoding heavy and light chains of mAb CO17-1AK were inserted into pBI121 plant expression vector. The cauliflower mosaic virus (CaMV) 35S promoter and alfalfa mosaic virus untranslated leader sequence (AMV) were inserted to be disposed in front of the heavy chain gene. In addition, the potato proteinase inhibitor II promoter (Pin2p) was inserted in front of the light chain gene to construct an expression cassette. The constructed heavy chain and light chain expression cassettes as such were treated with restriction enzymes HindIII and EcoRI, followed by its insertion into the plant expression vector pBI121.

The prepared plant expression vectors were introduced into Agrobacterium tumefaciens using electroporation, respectively. Agrobacterium retaining the inserted genes were then selected and cultured. The cultured Agrobacterium was inserted into the young leaves after formation of a cut with a size of 1-3 cm. The plant leaves were then transferred to solid plant medium, and then cultured on Murashige and Skoog solid medium (Dachfu, Haarlem, Netherland) supplemented with hormones, such as NAA (acetic acid) and BA (6-benzyl-amino-purine), and kanamycin (100 mg/L) until callus was generated. New transformant plants were generated 3-4 weeks after the culture.

### Example 2

Mating of Antigen-Expressing Plant and Antibody-Expressing Plant and Screening First-Generation Plant Simultaneously Expressing Traits of Parent-Generation

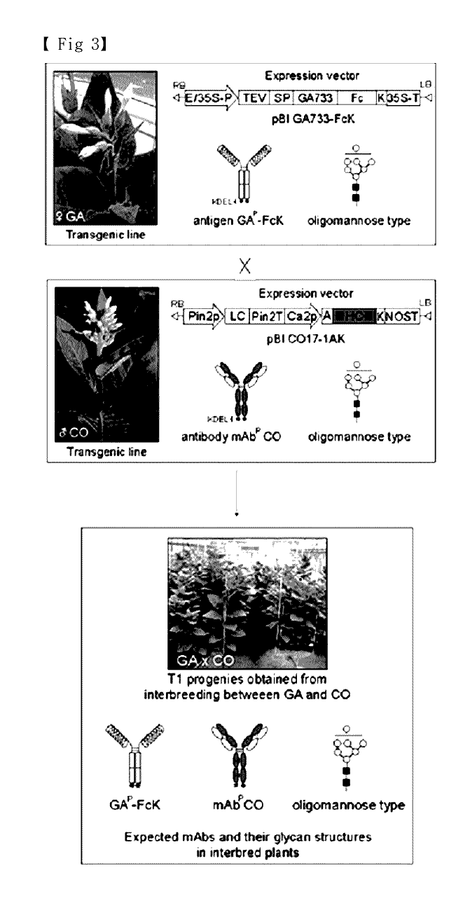

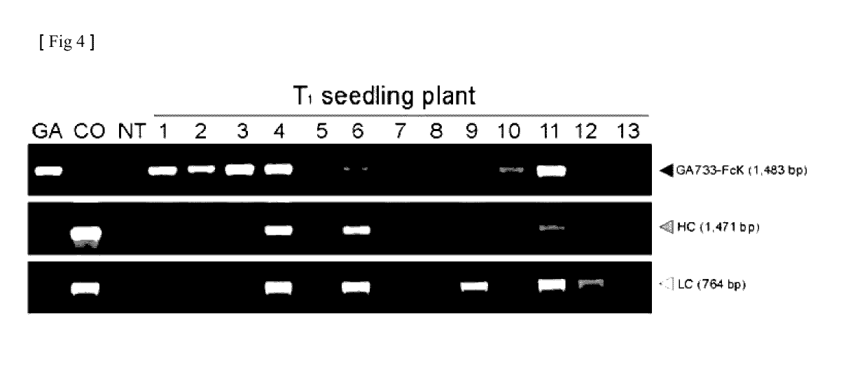

The cross-pollination was performed (see FIG. 3) by artificially placing the stamen of the colorectal cancer cell surface specific protein-Fc antibody (mAb CO17-1AK antibody) onto a flower bud of the plant expressing colorectal cancer cell surface specific protein-Fc (GA733-FcK antigen) produced from <Example 1>. The seeds obtained through the cross-pollination were germinated at 23° C. to grow a plant, thereby obtaining a total of 13 T1-generation (GA733-FcK×CO17-1AK) plants. The presence or absence of two genes in the T1-generation plants was confirmed using PCR method. The plants having two genes in each plant subject were selected and screened (see, FIG. 4). A specific experiment method was as follows.

Genomic DNA was separated and purified using Dneasy kit (Quiagen, Hilden, Germany) from leaves of the plant expressing the colorectal cancer cell surface specific protein-Fc (GA733-FcK antigen), the plant expressing the colorectal cancer cell surface specific protein-Fc antibody (mAb CO17-1AK), and the plant (GA733-FcK×CO17-1AK) obtained through the cross-pollination of the above two plants. The plant leaves were taken in approximately 90-100 g, instantly frozen in liquid nitrogen, and then pulverized. After the pulverization, pure plant genomic DNA was purified according to the method recommended by the Dneasy kit manufacturer. PCR was performed using each isolated genomic DNA as a template, a primer of colorectal cancer cell surface specific protein-Fc (GA733-FcK antigen), and primers of heavy chain and light chain of the colorectal cancer cell surface specific protein-Fc antibody (mAb CO17-1AK). The previously isolated genomic DNA (1 μl) and iTaq premix (Intron Biotechnol. Inc., Seongnam, Korea) were mixed, and forward primer 5′-GTCGACACGGCGACTTTTGCCGCAGCT-3′ (SEQ ID NO: 17) and reverse primer 5′-GAGTTCATCTTTACCCGGGGACAG-3 (SEQ ID NO: 18) of GA733-FcK were added at 10 pmol/μl. PCR conditions were as follows: 30 cycles of denaturation-annealing-elongation at 94° C. for 30 s, 67° C. for 30 s, and 72° C. for 30 s. In the same manner, each PCR was performed using forward primer 5′-ATGGAATGGAGCAGAGTCTTT-3′ (SEQ ID NO: 19) and reverse primer 5′-ATCGATTTTACCCGGAGTCCG-3 (SEQ ID NO: 20) of the heavy chain of mAb CO17-1AK and forward primer 5′-ATGGGCATCAAGATCGAATCA-3′ (SEQ ID NO: 21) and reverse primer 5′-ACACTCATTCCTGTTGAAGCT-3 (SEQ ID NO: 22) of the light chain of CO17-1AK.

As shown in FIG. 4, the results verified that both of GA733-FcK and CO17-1AK were expressed in plant NOs. 4, 6, and 11.

### Example 3

Verification on Gene Expression in Selected T1-Generation Plants

The expression of antigen and antibody for the plants selected in <Example 2> was investigated.

<3-1> Western Blot

100 mg of fresh leaves were taken from each of the transformed (or transgenic) plants GA733-FcK and CO17-1AK in <Example 1> and GA733-FcK×CO17-1AK (T1-generation plants) in <Example 2>, and put in 300 μl of 1×PBS KCl, Na₂HPO₄, KH₂PO₄), followed by sufficient pulverization. The supernatant of the pulverized leaves was subjected to electrophoresis on 10% SDS-PAGE gel. The supernatant was transferred to a nitrocellulose membrane, and then blocked with 5% skim milk (Fluka, Buchs, Switzerland) at 4° C. for 16 h. For secondary antibody treatment, anti-EpCAM/TROP1 (R&D system, Minneapolis, Minn.) and anti-mouse IgG H+L (Bethyl, Montgomerty, Tex.) diluted at a ratio of 1:5,000 were treated. Membrane washing was performed using 1×PBS (Tween 0.1%) buffer three times for 10 min each time. After the buffer was removed from the membrane, the membrane was reacted with Supersignal chemiluminescence substrate (Thermo, Fisher Scientific, Roskilde, Rosilde, Denmark), and then photosensitized on the X-ray film.

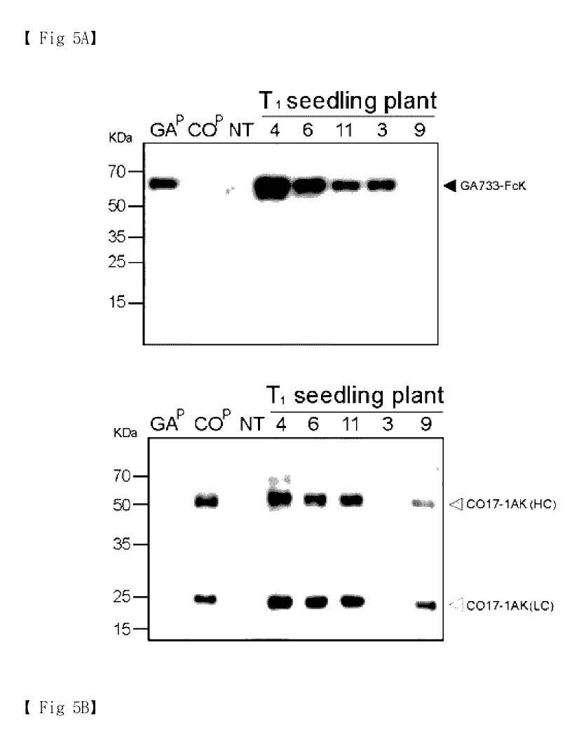

The western blot test verified that both of the antigen (FIG. 5a) and antibody (FIG. 5b) had high expression rates in T1-generation plants NOs. 4, 6, and 11.

<3-2> Electrophoresis and Two-Color Western Blot

Out of the plants confirmed to have expressed the antigen and the antibody in Example <3-1>, plant NO. 4 was grown in vivo condition (greenhouse). The leaves of the transformed (or transgenic) plants were purified, and then its protein molecular size was confirmed, while the plants expressing the two genes were confirmed through the two-color western blot. A specific experiment method was as follows.

Plant lines 4, 6, and 11 confirmed in vitro conditions were planted in the nursery bed soil (Sunshine Mix5, Agawam, Mass.). The temperature and humidity of the green house were 34° C. and 64% RH which are the average condition during July to September. When the plants were grown to adult plants and produced flowers, only leaves were collected and harvested, and then stored at −70° C. The collected leaves were used to purify antigen-antibody proteins. The plant purification was performed using protein G column (GE healthcare, Little Chalfont, United Kingdom). In each sample, GA^{M} is the chimeric antigen protein produced by the same method as described in <Example 1> using the GA733 protein and “‘Anti-Human EpCAM/TROP1 MAb [Clone 158210] (Mouse IgG2A, CATALOG# MAB960)” purchased from the R&D systems, and CO^{M} means mouse-derived mAb^{M} CO17-1A. GA^{P}(GA733^{P}-FcK), CO^{P}(mAb^{P} CO17-1AK), and GA^{P}×CO^{P}(GA733^{P}-FcK×mAb^{P}CO17-1AK) were the plants expressing a chimeric antigen and an antibody against the same, and a recombinant protein obtained from the plant prepared through cross-pollination of the plants as described in Examples 1 & 2. SDS-PAGE (sodium dodecyl sulfate-polyacrylamide gel electrophoresis) was made to 10% gel, and the respective protein samples were subjected to electrophoresis.

For the two-color western blot, 8 ul of the respective purified samples GA^{M} (chimeric antigen of GA733 and anti-Human EpCAM/TROP1 MAb), GA^{P} (GA733^{P}-FcK), CO^{M} (mAb^{M} CO17-1A), CO^{P} (mAb^{P} CO17-1AK), GA^{P}×CO^{P} (GA733^{P}-FcK×mAb^{P} CO17-1AK) at a concentration of 0.5 μg/μl were mixed with 2 μl of 5× loading buffer. Electrophoresis was performed using 10% SDS-PAGE, and the membrane was transferred to a nitrocellulose membrane, and then blocked with 5% skim milk (Fluka, Buchs, Switzerland) buffer at 4° C. for 16 h. For secondary antibody treatment, goat anti-human IRDye 800 CW (LI-COR, Lincoln, Nebr.) and goat anti-mouse IRDye 680 LT (LI-COR, Lincoln, Nebr.) were mixed with skim milk at a ratio of 1:15,000, followed by treatment at room temperature for 16 h. Membrane washing was performed using 1×PBS (Tween 0.1%) buffer three times for 10 min each time. The buffer of the membrane was removed, and then protein bands were confirmed using the infrared imaging system Odyssey detector (LI-COR, Lincoln, Nebr.).

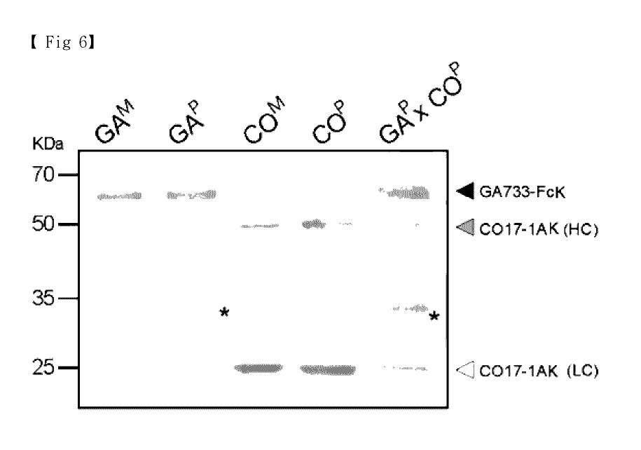

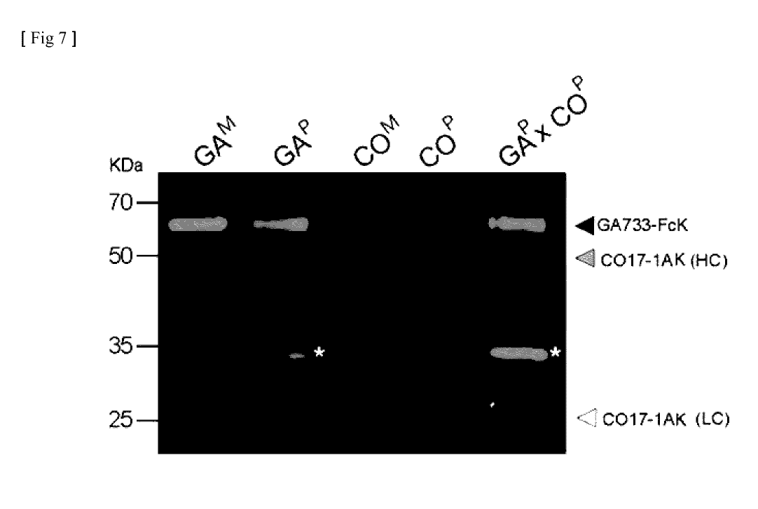

The results verified that two proteins GA733-FcK and CO17-1AK were purified in T1-generation plants using SDA-PAGE (see FIG. 6). In addition, it was verified through the two-color western blot that two proteins GA733-FcK and CO17-1AK were simultaneously expressed in samples purified from the T1-generation plants (see FIG. 7).

### Example 4

Confirmation of Morphology and Structure of Proteins

<4-1> Prediction of Structure of Protein Complex Through Sandwich ELISA

The sandwich ELISA was performed using the samples purified in Example <3-2>.

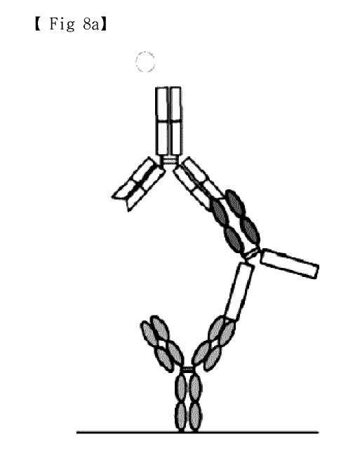

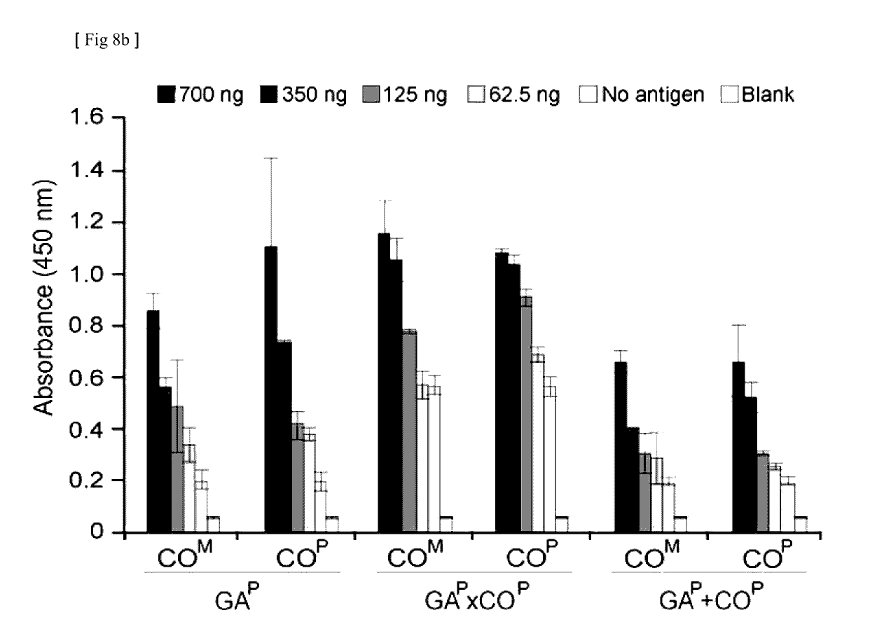

Specifically, 100 μl of CO^{M}(mAb^{M} CO17-1A) or CO^{P}(mAb^{P} CO17-1AK) as a capture antibody was dispensed at a concentration of 5 ng/μl in each well of the 96-well plat, and cultured at 4° C. overnight. In order to remove non-binding antibodies, the treated solution was removed from the well, and then the plate wells were washed three times with 1×PBS. In addition, 150 μl of 3% BSA solution was dispensed at 4° C. overnight. After the treated 3% BSA was removed, the wells were washed three times with 200 μl of 1×PBS. Antigens GA^{P}(GA733^{P}-FcK) and GA^{P}+CO^{P}(GA733^{P}-FcK+mAb^{P} CO17-AK, purified from the plants, and the same amount of proteins purified from the plants were mixed in vitro), and GA^{P}×CO^{P} (GA733^{P}-FcK×mAb^{P} CO17-1AK, protein purified from T1-generation plant NO. 4) was treated at 700 ng, 350 ng, 125 ng, and 62.5 ng on the samples, respectively, followed by incubation at 37° C. for 1½ hr. In addition, washing was repeated three times with 1×PBS. Anti-human Fc-HRP (Jackson ImmunoReseach Labs, west grove, PA) as a detection antibody and 3% BSA solution at a ratio of 1:10,000, were dispensed in 150 μl per each well, followed by incubation at room temperature for 2 h. After the incubation, each well was treated with TMB (3,3, 5,5-tetramethylbenzidine) substrate (KPL, Gaithersburg, Md., USA). In addition, the absorbance was confirmed at 450 nm. The binding of the capture antibody and the antigen (chimeric antigen according to the present invention, specifically GA733-FcK protein) and the binding form of the detection antibody recognizing the bound antigen-antibody complex are shown in FIG. 8a.

As a result, as shown in FIG. 8b, GA^{P}×CO^{P} showed higher absorbance than GA^{P} and GA^{P}+CO^{P}. Especially, the absorbance signal of GA^{P}+CO^{P} was smaller than that of GA^{P}, and this result was compared with the fact that GA^{P}×CO^{P} showed higher absorbance than GA^{P}, indicating that a large quaternary structure was not generated in GA^{P}+CO^{P}. Therefore, it was presumed that the antigen-antibody complex of the proteins purified from the T1-generation transformed (or transgenic) plants (especially, plant NO. 4) configures a stronger complex and forms a larger molecule than the antigen-antibody complex generated by the in vitro artificial binding of the antigen and antibody.

<4-2> Prediction of Structure of Protein Complex Through Surface Plasmon Resonance (SPR)

In order to validate that the antigen-antibody complex of the proteins purified from T1-generation transformed (or transgenic) plant (especially, transformed (or transgenic) plant NO, 4) configures a stronger complex and forms a large molecule, SPR was performed using GA or anti-GA antibody-coated SPR chip. Specifically, SPR was performed using ProteOn XPR36 surface instrument (Bio-Rad). GA^{M} (R&D systems) or CO^{M} was fixed to GLC sensor chip (Bio-Rad) using amine coupling chemistry according to the protocol provided by the manufacturer. The resonance unit (RU) was about 1,6001,800. The chip stabilization was performed by flowing PBS-T buffer at a flow rate of 100 μL/min for 60 s. Each sample (15 μg/mL) was allowed to flow on the receptor fixed at pH 6.0 at a flow rate of 50 μL/min at 25° C. After each measurement, the surface of the sensor chip was regenerated using phosphoric acid. In all experiments, data were 0 or were adjusted according to the standard channel. The dissociation and rate constant were calculated using Proteon Manager (Bio-Rad).

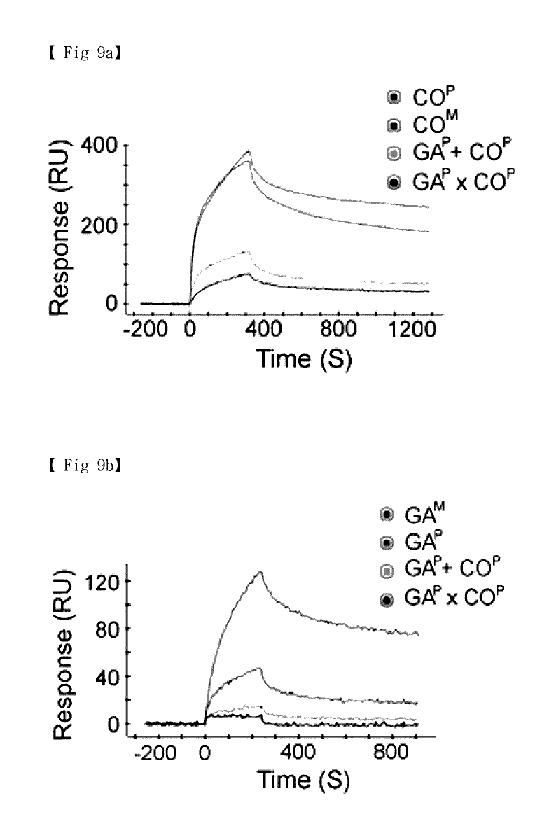

As a result, as shown in FIG. 9a, on the GA-coated SPR chip, the kinetic signals of GA^{P}×CO^{P} and GA^{P}+CO^{P} were significantly low compared with those of CO^{P} and CO^{M}. Further, as shown in FIG. 9b, on the anti-GA-antibody-coated SPR chip, the signal level of GA^{P}×CO^{P} was lower than that of GA^{P}+CO^{P}. These results support that the antigen-antibody complex configuring a large quaternary structure was generated in the T1-generation plants of the present invention.

<4-3> Electron Microscopic Observation

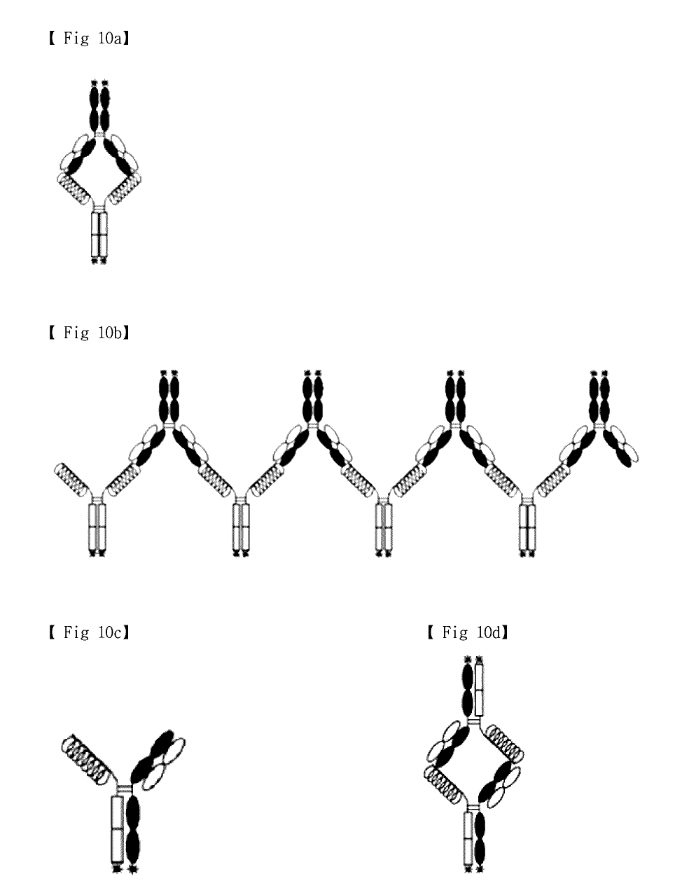

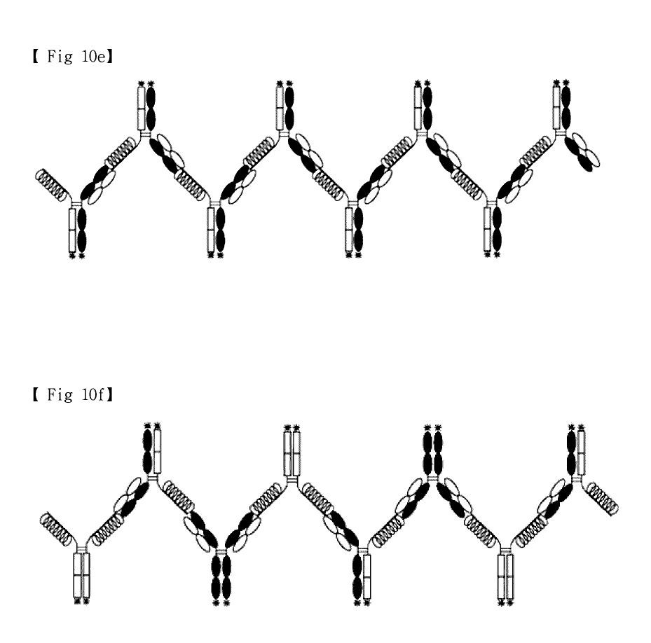

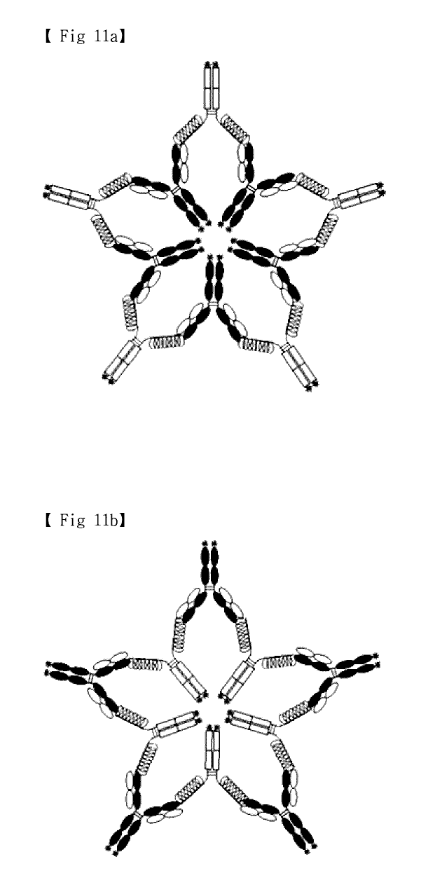

It was predicted from the results of Examples <4-1> and <4-2> that large quaternary structures shown in FIGS. 10 and 11 were generated in the T1-generation plants, and this prediction was confirmed. Specifically, for each of the protein samples obtained from the parent-generation antigen-expressing plant (GA733-FcK antigen) prepared in <Example 1> and offspring T1-generation prepared in <Example 2>, the protein structure and morphology were monitored by staining and electron microscope. Protein samples were incubated at 37° C. for 1 h. After the centrifugation, each sample was re-dispersed in PBS for preparing a specimen of transmission electron microscopy (TEM). The sample solution was loaded on a carbon film-coated TEM grid having hydrophilicity by glow ejection. After 90 s, an excessive sample solution was wiped out with distilled water. For negative staining, 1% uranyl acetate was loaded on a grid for 1 min, and then an excessive staining solution was wiped with filter paper. The samples were photographed by the bio-transmission electron microscope.

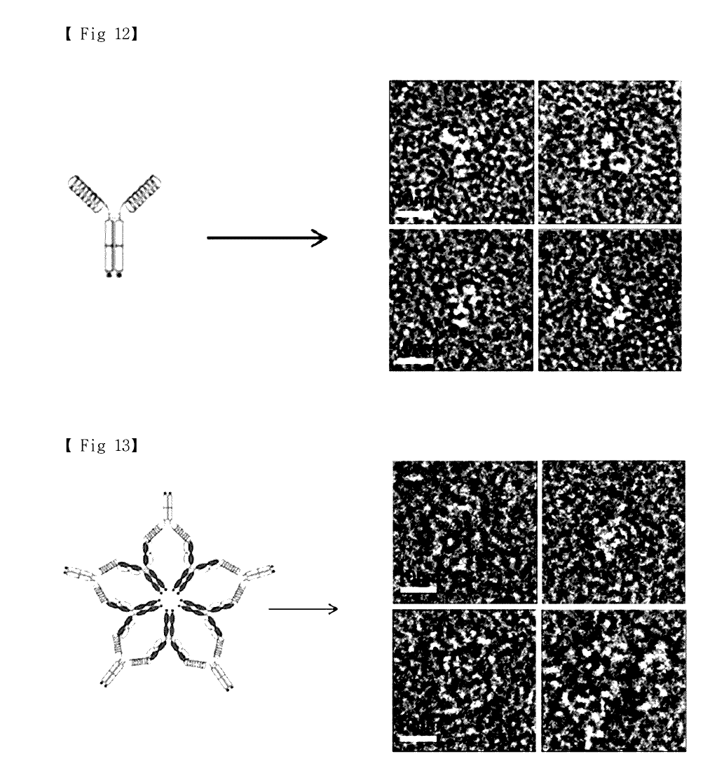

FIG. 12 shows a structure of GA733-FcK protein (antigen) expressed in the parent-generation plant, and FIG. 13 shows electron microscopic results of a structure of a protein sample obtained in T1-generation plant in which GA733-F cK and an antibody CO17-1AK against the same were simultaneously expressed. As shown in FIG. 12, the GA733-FcK protein (antigen) is observed in a Y-shape (˜15 nm) and various shapes, and an antigen protein existing alone was observed. Also, as shown in FIG. 13, a loop-shaped circular form (20-30 nm) shown in FIG. 11 was observed in the protein sample obtained from the T1-generation plant, while a ball-shaped form of 30 nm or larger and an aggregate of 30 nm or larger were also observed.

It can be seen from the above results that the antigen and the antibody configure a complex having a large quaternary structure with various shapes in the offspring-generation (A733-FcK×CO17-1AK) plant produced through cross-pollination of the plant expressing colorectal cell surface specific protein-Fc (GA733-FcK antigen) and the plant expressing colorectal cell surface specific protein-Fc antibody (mAb CO17-1AK antibody).

### Example 5

Measurement of Vaccine Effect

<5-1> Measurement of Immunization by Vaccination (Measurement of In Vivo Antibody Production)

The effect of vaccination was investigated by injecting four protein samples into mice.

Four protein samples used in the present test were as follows: GA^{M} (chimeric antigen protein produced by the same method as in <Example 1> using GA733 protein and “Anti-Human EpCAM/TROP1 MAb [Clone 158210](Mouse IgG2A, CATALOG#. MAB960)” marketed by R&D systems), GA^{P} (GA733^{P}-FcK), GA^{M}+CO^{M} (obtained by mixing in vitro the same amounts of proteins, a chimeric antigen of GA733 and anti-Human EpCAM/TROP1 MAb and mAb^{M} CO17-1A), GA^{P}×CO^{P} (GA733^{P}-FcK×mAb^{P} CO17-1AK)

Five mice per each group were used, and the four protein samples were injected without an immune adjuvant. 1×PBS was administered into a control group. After the injection of the samples, the serum of each group was obtained, and the amount of antibody produced in serum per each group was checked using a surface plasmon resonance (SPR) method as shown in Example <4-2>. Briefly speaking, for the surface plasmon resonance (SPR), a colorectal cancer candidate protein GA^{P} (GA733-FcK) was attached to a gold chip, and then 10 μl of the serum of each of the vaccinated mice was allowed to flow through the gold chip.

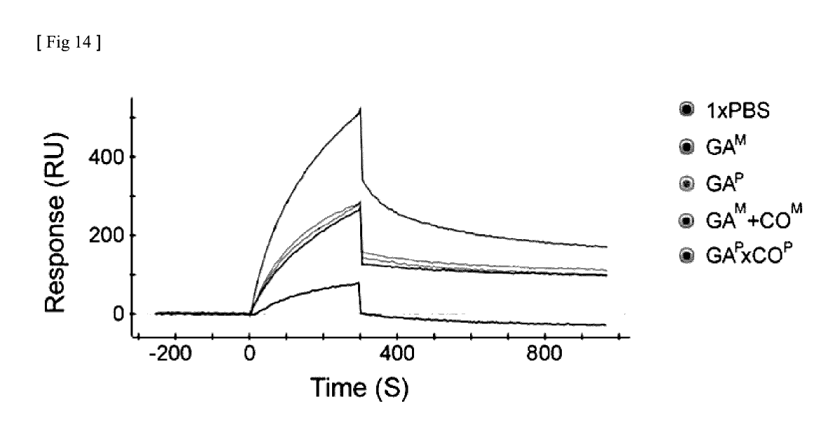

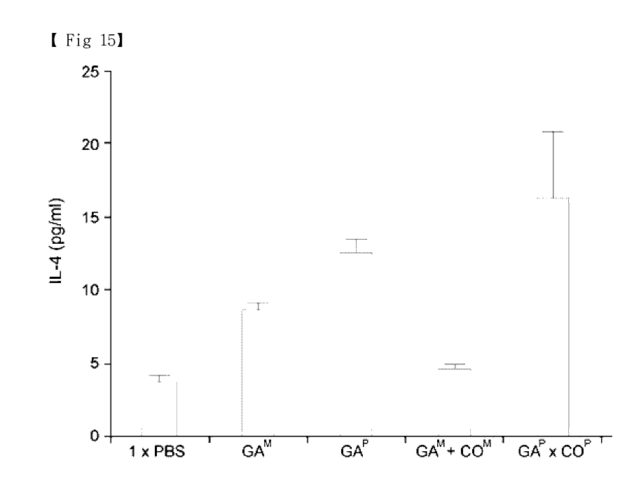

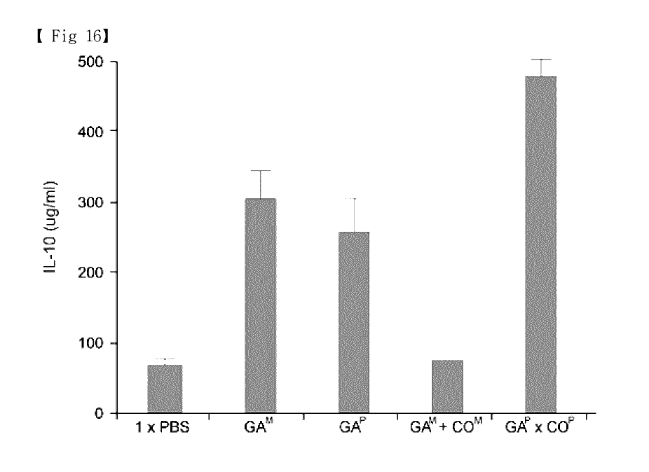

As a result of checking the difference between groups by measuring the amount of antibody produced in the serum of the mice, as shown in FIG. 14, it was verified that the serum of the mice injected with 1×PBS (negative control) showed the lowest signal while GA^{P}×CO^{P} showed a higher value compared with the other test groups, and thus GA^{P}×CO^{P} induced a higher immune response than any other vaccine candidate group. It was especially verified that GA^{P}×CO^{P}, which is the immunogenic complex obtained from the plant of the present invention, showed an excellent immunopotentiating effect, compared with the administration effect of GA^{M}+CO^{M}, which is the immune complex prepared in vitro. These results are due to the fact that the antigen-antibody complex produced by plant mating of the present invention configures a complex through stronger binding, compared with the antigen-antibody binding produced when the antibody and the antigen are placed in vitro at the same point in the prior art.

<5-2> Measurement of Immune Cell Activation (Measurement of Cytokine Production)

The spleen was extracted from each of the vaccinated mice in Example <5-1>, and disrupted together with media, and then dendritic cells and GA733-FcK as an antigen were co-cultured. The co-cultured flask was cultured at 37° C. for 3 days. After the culture, the activation of IL-4 and IL-10 was measured using FACS. The present test checked whether CD4+ of T cells was activated. CD4+ may be divided into classic Th1/Th2/Th17 responses, while IL-4 and IL-10 are factors included in Th2.

As a result, as shown in FIGS. 15 and 16, the spleen of the mice injected with GA^{P}×CO^{P} showed the highest IL-4 and IL-10 cytokine values compared with the mice immunized with 1×PBS, GA^{M}, GA^{P}, or GA^{M}+CO^{M}. These results indicate that T cell activation was increased in the mice injected with GA^{P}×CO^{P}. These results verified that the large molecule antigen-antibody complex of the present invention increases CD4+, and further influences the formation of antibodies.

<5-3> Comparison of In Vivo Cancer Growth Inhibitory Effect

Human colon cancer cells, SW 620 cells (1×10⁶) were intradermally (i.d.) inoculated in the back of 6-week age BALB nu/nu mice (three animals per each group, Japan SLC Inc., Hamamatsu, Shizuoka, Japan) to construct tumor xenograft mouse models. 40 μl of the serum obtained from each of BALB/c mice immunized with 1×PBS, GA^{M}, GA^{P}, GA^{M}+CO^{M}, or GA^{P}×CO^{P} was intraperitoneally injected into six groups of the tumor xenograft mouse models a total of four times every three days (administered at a total of 160 μl for 7 days). The positive control group was injected with 100 μg of purified mAb CO17-1A(CO^{M}). The growth of tumor, that is, the tumor volume was recorded on day 8, 10, 12, and 15 after the initial injection of cancer cells, and was calculated on the basis of three main diameters measured by graduated calipers by the following equation: (mm³)=width×length×height.

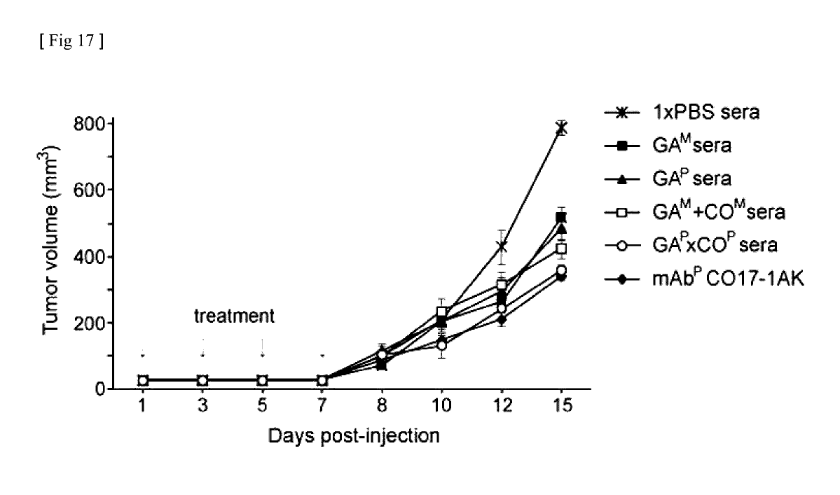

The test results are shown in FIG. 17. In nude mice injected with the serum obtained from BALB/c mice xenografted with SW 620 human colon cancer cells and immunized with 1×PBS, GA^{M}, GA^{P}, GA^{M}+CO^{M}, or GA^{P}×CO^{P}, tumor symptoms appeared on day 8 from the transplantation of cancer cells. Thereafter, the tumor size was abruptly grown in the 1×PBS treated group compared with the other test groups. The tumor was significantly quickly grown in the GA^{M} serum, GA^{P} serum, and GA^{M}+CO^{M} serum administered groups compared with the GA^{P}×CO^{P} serum or CO^{P} serum administered group. On day 15, the tumor size of the GA^{P}×CO^{P} administered group was significantly small compared with those of the other test groups. The tumor growth inhibitory effect in GA^{P}×CO^{P} administered group was similar to that in the CO^{P} administered group.

### Example 6

Analysis of Sugar Composition of First-Generation Protein

For comparison of N-glycan profile among GA^{P}, CO^{P} and GA^{P}×CO^{P}, mass analysis was performed.

The recombinant protein samples purified from the parent-generation (GA^{P}, CO^{P}) and T1-generation (GA^{P}×CO^{P}) plants were first digested into glycopeptides using pepsin. N-glycans were released from the glycopeptides using PNGas A (Roche), and the released N-glycans were purified using graphitized carbon resin from Carbograph (Alltech). The purified glycans were resuspended in a mixture of 90 μL dimethyl sulfoxide (DMSO), 2.7 μL of water, and 35 μL of iodomethane, and then solid phase permethylation was performed using a spin-column method (Goetz J A et al., 2009). The thus obtained permethylated glycans were mixed in equal volume with 10 mg/mL 2,5-dihydroxybenzoic acid solution (prepared in 1 mM sodium acetate solution). The resulting mixtures were applied onto a matrix-assisted laser-desorption-ionization (MALDI) MSP96 ground steel target plate and dried, followed by MALDI-TOF mass spectrometry. All mass spectra were acquired at a 20 kV accelerating voltage.

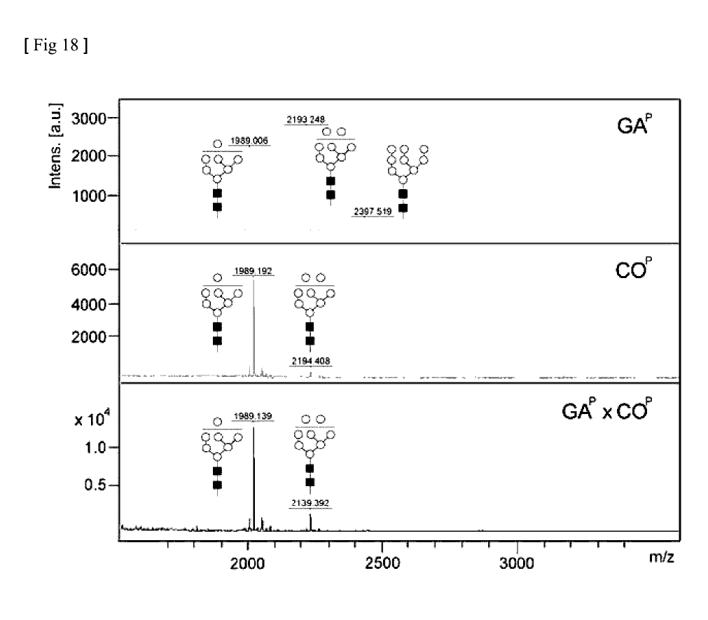

As shown n FIG. 18, the mass analysis results verified that oligomannose glycans (Man 79) were present in all the samples. It was verified that CO^{P} mainly has a Man 7 glycan structure while GA^{P} has a Man 79 oligomannoseglycan structure. Like CO^{P} and GA^{P}, GA^{P}×CO^{P} also had an oligomannoseglycan structure. Further, the relative ratio (4:1) of Man 7 and Man 9 in GA^{P}×CO^{P} was similar to a sum of those in CO^{P} and GA^{P}. Therefore, it can be seen that the immune complex expressed in the T1 generation contains almost the same glycan structure as in the proteins of the parent generation.

### INDUSTRIAL APPLICABILITY

As set forth above, the present invention relates to a method for preparing a transformed (or transgenic) plant producing an immunogenic complex protein and an immunogenic complex protein obtained therefrom and, more specifically, to a method for preparing a transformed (or transgenic) plant producing an immunogenic complex protein, the method comprising: (a) preparing a transformed (or transgenic) plant expressing an antigen; (b) preparing a transformed (or transgenic) plant expressing an antibody specific to the antigen in step (a); and (c) mating the plants in steps (a) and (b) to prepare a mated plant, to a plant produced by the method, and to an immunogenic complex protein obtained from the plant.

Through the method for preparing a transformed (or transgenic) plant, comprising steps (a) to (c), and the transformed (or transgenic) plant produced by the method, immunogenic complex proteins can be mass-produced safely and economically. Furthermore, the immunogenic complex protein (antigen-antibody complex) obtained from the plant has a large quaternary structure, thereby having an excellent effect in boosting immune response, and thus exhibits an remarkable capability in producing antibodies in a host animal even without using an immune adjuvant.

What is claimed is:

1. A method for preparing a transformed plant producing an immunogenic complex protein, the method comprising:
(a) preparing a transformed plant expressing an antigen;
(b) preparing a transformed plant expressing an antibody specific to the antigen in step (a); and
(c) mating the plants in steps (a) and (b) to prepare a mated plant.

2. The method of claim 1, wherein the antigen is a chimeric antigen comprising (i) and (ii) below:
(i) an immune response domain comprising an antigenic protein; and
(ii) a target binding domain comprising an Fc antibody fragment.

3. The method of claim 2, wherein the target binding domain (ii) further comprises a hinge region of an immunoglobulin, a heavy chain CH1 domain, or a linker.

4. The method of claim 1, wherein the antigen in step (a) is a colorectal cancer cell surface protein GA733.

5. The method of claim 2, wherein the Fc antibody fragment (ii) comprises a hinge region of IgG specific to GA733, a CH2 domain, and a CH3 domain.

6. The method of claim 1, wherein the antigen in step (a) is GA733-FcK chimeric antigen represented by SEQ ID NO: 9.

7. The method of claim 1, wherein the antibody in step (b) is a monoclonal antibody.

8. The method of claim 1, wherein the antibody in step (b) is a bivalent antibody.

9. The method of claim 1, wherein the antibody in step (b) is an antibody specific to GA733-FcK chimeric antigen, the antibody comprising a heavy chain represented by SEQ ID NO: 12 and a light chain represented by SEQ ID NO: 13.

10. The method of claim 1, wherein the antigen in step (a) and the antibody in step (b) comprise an endoplasmic reticulum retention signal sequence.

11. The method of claim 1, wherein the plant is Nicotiana tabacum.

12. A plant producing an immunogenic complex protein, the plant being prepared by the method of claim 1.

13. The method of claim 12, wherein the immunogenic complex protein is an antigen-antibody complex of GA733-FcK chimeric antigen and an antibody specific thereto.

14. An immunogenic complex protein obtained from the plant of claim 12.

15. The method of claim 14, wherein the immunogenic complex protein is at least one selected from the group consisting of a chimeric antigen-antibody monomolecular form, a pentameric form obtained by polymerizing the chimeric antigen-antibody monomolecular monomers, and a linear structure obtained by cross-linking the chimeric antigen and antibody.

16. A vaccine composition comprising the immunogenic complex protein of claim 14 and a pharmaceutically acceptable carrier or diluent.

17. The immunogenic complex protein of claim 14 for preparing vaccine.

18. A method for immunization, the method comprising administering an effective amount of the immunogenic complex protein of claim 14 to a subject in need thereof.
